SEQUENCE LISTING (7 sequences)

SEQ ID NO: 1 - DNA, 75 bp, Artificial Sequence
  STAT5 Response Element
agttctgagaaaagtagttctgagaaaagtagttctgagaaaagtagttctgagaaaagtagttctgagaaaagt

SEQ ID NO: 2 - DNA, 1650 bp, Artificial Sequence
  DNA encoding Luc2P codon-optimized for expression in human cells
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtg

SEQ ID NO: 3 - DNA, 32 bp, Artificial Sequence
  Mini promoter element
tagagggtatataatggaagctcgacttccag

SEQ ID NO: 4 - DNA, 120 bp, Artificial Sequence
  DNA encodes PEST degradation sequence from mouse ornithine codon-optimized for expression in human cells
tctcacggcttccctcccgaggtggaggagcaggccgccggcaccctgcccatgagctgcgcccaggagagcggcatggatagacaccctgctgcttgcgccagcgccaggatcaacgtc

SEQ ID NO: 5 - DNA, 6079 bp, Artificial Sequence
  JX206457.1 Reporter vector pGL4.52[luc2P/STAT5 RE/Hygro], complete sequence
actcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagctcagttctgagaaaagtagttctgagaaaagtagttctgagaaaagtagttctgagaaaagtagttctgagaaaagtctcgaggatatcaagatctggcctcggcggccaagcttagacactagagggtatataatggaagctcgacttccagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattctcacggcttccctcccgaggtggaggagcaggccgccggcaccctgcccatgagctgcgcccaggagagcggcatggatagacaccctgctgcttgcgccagcgccaggatcaacgtctaaggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaaactctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcat

SEQ ID NO: 6 - protein, 133 aa, Artificial Sequence [sequence omitted]

SEQ ID NO: 7 - protein, 132 aa, Artificial sequence
  Aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2024/0255492 A1
(43) Pub. Date: Aug. 1, 2024

(54) FUNCTIONAL CELL-BASED POTENCY ASSAY FOR MEASURING BIOLOGICAL ACTIVITY OF INTERLEUKIN 2 (IL-2) ANALOGS

(71) Applicant: MERCK SHARP & DOHME LLC, RAHWAY, NJ (US)

(72) Inventors: DANILA G. GIACONE, BOUND BROOK, NJ (US); DAVID P. SANDEN, SAN FRANCISCO, CA (US); AARRON WILLINGHAM, MOUNTAIN VIEW, CA (US); JUNMING YIE, WARREN, NJ (US)

(73) Assignee: MERCK SHARP & DOHME LLC, RAHWAY, NJ (US)

(21) Appl. No.: 18/564,642

(22) PCT Filed: Jun. 2, 2022
(86) PCT No.: PCT/US2022/031872

§ 371 Date: Nov. 28, 2023

Related U.S. Application Data
(60) Provisional application No. 63/330,817, filed on Apr. 14, 2022.
(60) Provisional application No. 63/208,223, filed on Jun. 8, 2021.

(51) Int. Cl. G01N 33/50; C07K 14/55; C12N 5/0783; C12Q 1/66; G01N 33/68

(52) U.S. Cl. CPC G01N 33/505; C07K 14/55; C12N 5/0636; C12Q 1/66; G01N 33/6869; C12N 2510/00; G01N 2333/55; G01N 2500/10

(57) ABSTRACT

A functional cell-based potency assay for measuring the biological activity of IL-2 mutants with biased activity for the IL-2 receptor beta-gamma complex is described. In particular, the present invention relates to a Kit225 cell line that lacks expression of the IL-2 receptor alpha and its use in said functional cell-based potency assay.

### BACKGROUND OF THE INVENTION

### (1) Field of the Invention

The present invention relates to a functional cell-based potency assay for measuring the biological activity of IL-2 mutants with biased activity for the IL-2 receptor beta-gamma complex. In particular, the present invention relates to a Kit225 cell line that lacks expression of the IL-2 alpha receptor and its use in said functional cell-based potency assay.

### (2) Description of Related Art

Interleukin-2 (IL-2) is a key driver of many immunological processes, including the differentiation, activation, proliferation, and survival of the cells which provide anti-tumor immunity, including effector CD8⁺ T cells and NK cells (Mitra & Leonard, J. Leukoc. Biol. 103(4): 643-655 (2018)). Another important function of IL-2 is the contraction of immune responses through triggering activation-induced cell death and the expansion and activation of regulatory T cells (T_{regs}) (Boyman & Sprent, Nat. Rev. Immunol. 12: 180-190 (2012)). The effects of IL-2 are mediated by a complex receptor system comprised of three protein subunits: IL-2Rα (CD25), IL-2Rβ (CD122), and the common gamma chain γ (CD132) (Taniguchi & Minami, Cell, v73, 5-8 (1993)). IL-2Rα binds IL-2 with low affinity (no signal transduction). IL-2Rβ and IL-2Rγ form an intermediate affinity dimeric receptor IL-2Rβγ with an affinity of about Kd, 10⁻⁹ M, which is expressed on CD8 T cells and NK cells. IL-2Rα, IL-2Rβ, and IL-2Rγ together form the high affinity trimer receptor IL-2Rαβγ with an affinity of about Kd, 10-11 M, that binds IL-2 with high affinity and is expressed on regulatory T cells (T_{regs}), activated T cells, and endothelial cells. Due to this differential affinity, IL-2Rαβγ expressing cells will preferentially bind IL-2. A high dose of IL-2 activates the By dimer, resulting in activation of the immune response. However, a high dose of IL-2 also activates the αβγ trimer on T_{regs}, which suppresses activation of the immune response and may lead to tolerance of tumor antigens. Binding of IL-2 to either IL-2Rβγ or IL-2Rαβγ induces multiple signaling pathways and the transcription of target genes (Spolski et al., Nat Rev Immunol. 18: 648-659 (2018)). These pathways include the Jak/Stat pathway, the MAPK pathway and the PI3K pathway. T_{regs} are 100-fold more sensitive to IL-2 due to expression of the high affinity IL-2 receptor complex consisting of the IL-2Rα, β, and γ chains. In contrast to T_{regs}, effector T cells and NK cells primarily express an intermediate affinity receptor consisting of β and γ chains and are less sensitive to IL-2.

IL-2 is an approved immunotherapy that has shown clinical efficacy in a small subset of patients, with long term responses, including cures. IL-2 was the first cytokine, and immunotherapy, to be used successfully to treat cancer. In 1992, aldesleukin, a non-glycosylated human recombinant IL-2 analog (des-alanyl-1, serine-125 human IL-2), was approved by the U.S. Food and Drug Administration (FDA) for the treatment of Renal Cell Carcinoma (RCC) and Metastatic Melanoma. However, the use of IL-2 (Proleukin®, aldesleukin) as a therapeutic has been hampered by sometimes severe toxicity characterized by vascular leak syndrome (VLS). In light of the toxicity, there has been considerable interest in engineering an IL-2 analogue that selectively agonizes the IL-2Rβγ complex, generates fewer T_{regs}, which leads to a more favorable CD8: Treg ratio, thereby providing improved anti-tumor immunity. See, for example, U.S. Pat. Nos. 10,610,571; 9,861,705; 9,428,567; 9,266,938; 9,206,243; Int'l. Pat. Application Nos. WO9320849; WO2005086751; WO2008003473; WO2010085495; WO2019125732; WO2019185705; WO2019185706; WO2019028425; WO2020163532; WO2021030706; Chinese Pat. Application No. CN104231068; Suave et al. PNAS USA 88: 4636 (1991); and, Vazquez-Lombardi et al. Nat Comm. 8: Art. No. 15373 (2017). However, using functional cell-based assays for measuring the potency of IL-2 analogs with various mutations for enhancing selectivity for IL-2Rβγ complex and comparing to the potency of wild-type IL-2 has been difficult because of the presence of IL-2Rαβγ complexes in the cell-based assays. Therefore, there is a need for a functional cell-based assay that enables measuring and comparing IL-2 analog potencies without the interference of IL-2Rαβγ complexes.

### BRIEF SUMMARY OF THE INVENTION

The present invention provides a functional cell-based assay that can give a quantitative assessment of IL-2 mediated cellular signaling and the relative activity of engineered IL-2 analogs biased for the IL-2Rβγ complex compared to non-IL-2Rβγ biased IL-2 analogs in a cell environment that lacks expression of IL-2Rαβγ complexes. A cell line that expresses CD25, CD122, and CD132 was engineered to lack expression of CD25 and to comprise a signal transducer and activator of transcription 5 (STAT5) responsive reporter system comprising five copies of a STAT5 response element operably linked to a detectable polypeptide reporter. The engineered cell line expresses the IL-2Rβγ complex and not the IL-2Rαβγ complex. The present invention enables the comparison of the relative impact of PEGylation and IL-2Rα blocking on IL-2 potency.

The present invention provides a method for determining the potency of an IL-2 analog biased for the IL-2Rβγ complex over the IL-2Rαβγ complex, comprising (a) providing (i) a cell line that expresses an IL-2Rβγ complex without expression of an IL-2Rαβγ complex and a STAT5 signaling transduction pathway reporter comprising a STAT5 response element and promoter linked to an open reading frame encoding a detectable polypeptide, and (ii) serial dilutions of an IL-2 analog biased for the IL-2Rβγ complex; (b) contacting each serial dilution of the IL-2 analog biased for the IL-2Rβγ complex with an aliquot of the cell line to provide a plurality of cultures; (c) incubating the cultures for a time sufficient to enable expression of the detectable polypeptide over time; and (d) measuring expression of the detectable polypeptide to determine the potency of the IL-2 analog biased for the IL-2Rβγ complex.

In a further embodiment, the method further comprises comparing the potency of the IL-2 analog biased for the IL-2Rβγ complex to the potency of a control IL-2 analog, which comprises an IL-2 analog capable of binding to an IL-2Rαβγ complex.

In a further embodiment, the potency of the IL-2 analog capable of binding to an IL-2Rαβγ complex is determined by (e) providing the cell line of step (a) above and serial dilutions of the IL-2 analog capable of binding to an IL-2Rαβγ complex; (f) contacting each serial dilution of the IL-2 analog capable of binding to an IL-2Rαβγ complex with an aliquot of the cell line to provide a plurality of cultures; (g) incubating the cultures for a time sufficient for expression of the detectable polypeptide over time; and (h) measuring expression of the detectable polypeptide to determine the potency of the IL-2 analog capable of binding to an IL-2Rαβγ complex.

In a further embodiment, the cell line comprises Kit225 cells, which have been modified to lack expression of the CD25 gene.

In a further embodiment, the detectable polypeptide is a luciferase polypeptide.

In a further embodiment, the STAT5 response element comprises one or more copies of the nucleotide sequence TCCNNNGAA wherein each N is any nucleotide or the nucleotide sequence set forth in SEQ ID NO: 1.

In a further embodiment, the IL-2 analog capable of binding to an IL-2Rαβγ complex comprises aldesleukin.

In a further embodiment, the IL-2 analog biased for the IL-2Rβγ complex comprises at least one amino acid substitution or deletion that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises at least one non-natural amino acid substitution that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises one or more substitutions or deletions at a position selected from the group consisting of E15, H16, L19, D20, K34, T36, R37, T40, F41, K42, F43, Y44, E60, E61, K63, P64, E67, L71, D84, N88, V91, M103, C104, Y106, Q126, T123, and 1129, wherein the amino acid positions correspond to the positions set forth in the amino acid sequence of SEQ ID NO: 6.

In a further embodiment, the non-natural amino acid is conjugated to a hydrophilic or hydrophobic polymer.

In a further embodiment, the STAT5 signaling transduction pathway reporter is provided by an expression vector comprising a nucleic acid molecule comprising a STAT5 response element and promoter linked to an open reading frame encoding a detectable polypeptide. In a particular embodiment, the expression vector or fragment thereof is integrated into the genome of the cells comprising the cell line. In a particular embodiment, the expression vector persists in an autonomous state in the cells comprising the cell line.

The present invention further provides a method for producing an IL-2 analog biased for the IL-2Rβγ complex over the IL-2Rαβγ complex, comprising (a) providing an IL-2 analog capable of binding the IL-2Rαβγ complex; (b) substituting one or more amino acids of the IL-2 analog that are at the interface between the IL-2 and the IL-2Rα in the IL-2Rαβγ complex with a natural amino acid or non-natural amino acid to provide an IL-2 analog biased for the IL-2Rβγ complex; (c) making a serial dilution of the IL-2 analog biased for the IL-2Rβγ complex; (d) contacting each serial dilution of the IL-2 analog biased for the IL-2Rβγ complex with an aliquot of a cell line that expresses (i) the IL-2Rβγ complex without expression of the IL-2Rαβγ complex and (ii) a STAT5 signaling transduction pathway reporter comprising a STAT5 response element and promoter linked to an open reading frame encoding a detectable polypeptide, to provide a plurality of cultures; (e) incubating the cultures for a time sufficient for expression of the detectable polypeptide over time; (f) measuring expression of the detectable polypeptide to determine the potency of the IL-2 analog biased for the IL-2Rβγ complex; and (g) isolating the IL-2 analog biased for the IL-2Rβγ complex if the potency of the IL-2 analog biased for the IL-2Rβγ complex has a potency within a predetermined range.

In a further embodiment, the potency is substantially the same as the potency of an IL-2 analog capable of binding the IL-2Rαβγ complex.

In a further embodiment, the potency of the IL-2 analog capable of binding the IL-2Rαβγ complex is determined by (h) making a serial dilution of the IL-2 analog capable of binding the IL-2Rαβγ complex; (i) contacting each serial dilution of the IL-2 analog capable of binding the IL-2Rαβγ complex with an aliquot of a cell line that expresses (x) the IL-2Rβγ complex without also expressing an IL-2Rαβγ complex and (y) a STAT5 signaling transduction pathway reporter comprising a STAT5 response element and promoter linked to an open reading frame encoding a detectable polypeptide, to provide a plurality of cultures; (j) incubating the cultures for a time sufficient for expression of the detectable polypeptide over time; and (k) measuring expression of the detectable polypeptide to determine the potency of the IL-2 analog capable of binding the IL-2Rαβγ complex.

In a further embodiment, the IL-2 analog capable of binding the IL-2Rαβγ complex comprises aldesleukin.

In a further embodiment, the non-natural amino acid is conjugated to a hydrophilic or hydrophobic polymer.

In a further embodiment, the cell line comprises Kit225 cells, which have been modified to lack expression of the CD25 gene.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a further embodiment, the STAT5 signaling transduction pathway reporter is provided by an expression vector comprising a nucleic acid molecule comprising a STAT5 response element and promoter linked to an open reading frame encoding a detectable polypeptide. In a particular embodiment, the expression vector or fragment thereof is integrated into the genome of the cells comprising the cell line. In a particular embodiment, the expression vector persists in an autonomous state in the cells comprising the cell line.

A manufacturing process for producing a batch of an interleukin 2 (IL-2) analog biased for the IL-2 receptor beta-gamma (IL-2Rβγ) complex comprising the steps of: (a) synthesizing the IL-2 analog biased for the IL-2Rβγ complex; (b) purifying the IL-2 biased for the IL-2Rβγ complex; (c) formulating the IL-2 biased for the IL-2Rβγ complex into a batch; (d) obtaining a sample of the IL-2 analog biased for the IL-2Rβγ from the batch; (e) making a serial dilution of the IL-2 analog biased for the IL-2Rβγ complex; (f) contacting each serial dilution of the IL-2 analog biased for the IL-2Rβγ complex with an aliquot of a cell line that expresses (i) the IL-2Rβγ complex without expression of the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures; (g) incubating the cultures for a time sufficient to enable expression of the detectable polypeptide over time; and (h) measuring expression of the detectable polypeptide to determine the potency of the IL-2 analog biased for the IL-2Rβγ complex. In particular embodiments, the batch is a production batch.

In a further embodiment, the cell line comprises Kit225 cells, which have been modified to lack expression of the CD25 gene.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

The present further provides a Kit225 cell modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a particular embodiment, the nucleic acid molecule is integrated into the genome of the Kit225 cell. In a particular embodiment, the nucleic acid molecule persists in an autonomous state in the Kit225 cell, e.g., in a plasmid capable of replicating and being maintained in a eukaryote cell.

The present invention further provides a cell line comprising Kit225 cells modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a particular embodiment, the nucleic acid molecule is integrated into the genome of the Kit225 cells comprising the cell line. In a particular embodiment, the nucleic acid molecule persists in an autonomous state in the Kit225 cells comprising the cell line, e.g., in a plasmid capable of replicating and being maintained in a eukaryote cell.

In a further embodiment, the STAT5 signaling transduction pathway reporter is provided by an expression vector comprising a nucleic acid molecule comprising a STAT5 response element and promoter linked to an open reading frame encoding a detectable polypeptide. In a particular embodiment, the expression vector or fragment thereof is integrated into the genome of the cells comprising the cell line. In a particular embodiment, the expression vector persists in an autonomous state in the cells comprising the cell line.

The present invention further provides a manufacturing process for producing a batch of an IL-2 analog biased for the IL-2Rβγ complex comprising the steps of synthesizing the IL-2 analog biased for the IL-2Rβγ complex, purifying the IL-2 biased for the IL-2Rβγ complex, and formulating the IL-2 biased for the IL-2Rβγ complex into a batch, wherein the improvement comprises (a) obtaining a sample of the IL-2 analog biased for the IL-2Rβγ from the manufacturing process; (c) making a serial dilution of the IL-2 analog biased for the IL-2Rβγ complex; (d) contacting each serial dilution of the IL-2 analog biased for the IL-2Rβγ complex with an aliquot of a cell line that expresses (i) the IL-2Rβγ complex without expression of the IL-2Rαβγ complex and (ii) a STAT5 signaling transduction pathway reporter, which comprises a STAT5 response element and promoter linked to an open reading frame encoding a detectable polypeptide, to provide a plurality of cultures; (e) incubating the cultures for a time sufficient to enable expression of the detectable polypeptide over time; and (f) measuring expression of the detectable polypeptide to determine the potency of the IL-2 analog biased for the IL-2Rβγ complex. In particular embodiments, the batch is a production batch.

In a further embodiment, the cell line comprises Kit225 cells, which have been modified to lack expression of the CD25 gene.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

The present further provides a Kit225 cell modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a particular embodiment, the nucleic acid molecule is integrated into the genome of the Kit225 cell. In a particular embodiment, the nucleic acid molecule persists in an autonomous state in the Kit225 cell, e.g., in a plasmid capable of replicating and being maintained in a eukaryote cell.

The present invention further provides a cell line comprising Kit225 cells modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a particular embodiment, the nucleic acid molecule is integrated into the genome of the Kit225 cells comprising the cell line. In a particular embodiment, the nucleic acid molecule persists in an autonomous state in the Kit225 cells comprising the cell line, e.g., in a plasmid capable of replicating and being maintained in a eukaryote cell.

In a further embodiment, the STAT5 signaling transduction pathway reporter is provided by an expression vector comprising a nucleic acid molecule comprising a STAT5 response element and promoter linked to an open reading frame encoding a detectable polypeptide. In a particular embodiment, the expression vector or fragment thereof is integrated into the genome of the cells comprising the cell line. In a particular embodiment, the expression vector persists in an autonomous state in the cells comprising the cell line.

The present further provides a method for producing a modified Kit225 cell that lacks expression of the CD25 gene and comprises a nucleic acid molecule comprising a STAT5 signaling transduction pathway reporter comprising (a) the steps of deleting or disrupting the CD25 gene of a Kit225 cell to produce a Kit225 cell that lacks CD25 expression and transfecting said Kit225 cell that lacks CD25 expression with an expression vector comprising a STAT5 response element and promoter operably linked to an open reading frame encoding a detectable polypeptide to produce the modified Kit225 cell; or, (b) the steps of transfecting a Kit225 cell with an expression vector that comprises a STAT5 response element and promoter operably linked to an open reading frame encoding a detectable polypeptide and then deleting or disrupting the CD25 gene of said Kit225 cell to produce the modified Kit225 cell.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

The present invention further provides a method for determining the potency of an interleukin 2 (IL-2) analog, comprising: (a) providing (i) a cell line that expresses an IL-2Rαβγ complex and a signal transducer and activator of transcription 5 (STAT5) signaling transduction pathway reporter comprising a STAT5 response element and a promoter linked to an open reading frame encoding a detectable polypeptide, and (ii) serial dilutions of an IL-2 analog; (b) contacting each serial dilution of the IL-2 analog with an aliquot of the cell line to provide a plurality of cultures; (c) incubating the cultures for a time sufficient to enable expression of the detectable polypeptide over time; and (d) measuring expression of the detectable polypeptide to determine the potency of the IL-2 analog.

In a further embodiment, the method further comprises comparing the potency of the IL-2 analog to the potency of a control IL-2 analog.

In a further embodiment, the potency of the control IL-2 analog is determined by (e) providing the cell line of step (a) and serial dilutions of the control IL-2 analog; (f) contacting each serial dilution of the control IL-2 analog with an aliquot of the cell line to provide a plurality of cultures; (g) incubating the cultures for a time sufficient for expression of the detectable polypeptide over time; and (h) measuring expression of the detectable polypeptide to determine the potency of the control IL-2 analog.

In a further embodiment, the cell line comprises Kit225 cells.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

In a further embodiment, the control IL-2 analog comprises aldesleukin.

The present invention further provides a manufacturing process for producing a production batch of an interleukin 2 (IL-2) analog comprising the steps of: (a) synthesizing the IL-2 analog; (b) purifying the IL-2 analog; (c) formulating the IL-2 analog into a production batch; (d) obtaining a sample of the IL-2 analog from the production batch; (e) making a serial dilution of the IL-2 analog; (f) contacting each serial dilution of the IL-2 analog with an aliquot of a cell line that expresses (i) the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures; (g) incubating the cultures for a time sufficient for expression of the detectable polypeptide over time; and (f) measuring expression of the detectable polypeptide to determine the potency of the IL-2 analog biased.

In a further embodiment, the cell line comprises Kit225 cells.

In a further embodiment, the detectable polypeptide is a luciferase polypeptide.

In a further embodiment, the STAT5 response element comprises the nucleotide sequence set forth in SEQ ID NO: 1 or one or more copies of the nucleotide sequence TCCNNNGAA wherein each N is independently any nucleotide.

The present invention further provides a Kit225 cell comprising a nucleic acid molecule comprising a signal transducer and activator of transcription 5 (STAT5) response element and promoter operably linked to an open reading frame encoding a detectable polypeptide. In a further embodiment, the detectable polypeptide is a luciferase polypeptide. In a further embodiment, the STAT5 response element comprises the nucleotide sequence set forth in SEQ ID NO: 1 or one or more copies of the nucleotide sequence TCCNNNGAA wherein each N is independently any nucleotide.

The present invention further provides cell line comprising Kit225 cells comprising a nucleic acid molecule comprising a signal transducer and activator of transcription 5 (STAT5) response element and promoter operably linked to an open reading frame encoding a detectable polypeptide. In a further embodiment, the detectable polypeptide is a luciferase polypeptide. In a further embodiment, the STAT5 response element comprises the nucleotide sequence set forth in SEQ ID NO: 1 or one or more copies of the nucleotide sequence TCCNNNGAA wherein each N is independently any nucleotide.

### BRIEF DESCRIPTION OF THE DRAWINGS

FIGS. 1A-1B: Development of Kit225 STAT5Luc stable cell line. FIG. 1A. Several cell lines were tested to generate a strong pSTAT5 signal when treated with IL-2. Three cell lines, JVM-2, Kit225, and NK92 cells were evaluated. Phospho-STAT5 was detected using ELISA assay and reported as RLU (relative light units). Kit225 showed strong induction on STAT5 phosphorylation by IL-2. FIG. 1B. Dose response curve of IL-2 (aldesleukin) using the engineered Kit225 STAT5-Luc #8 cell line.

FIGS. 2A-2H. Development of CD25 K/O Kit225 STAT5Luc stable cell line. FIG. 2A: CD25 expression profile of Kit225STAT5Luc #8 Cell Line by FACS. FIG. 2B: Shows a Fluorescence-Activated Cell Sorting (FACS) profile of the first CRISPR-Cas 9 Experiment Utilizing Parental Kit225Stat5Luc with 3 Individual gRNAs. FIG. 2C: Shows a FACS profile of the CD25 expression profile of CRISPR-Cas9 CD25 K/O Pool 2-1 vs “Parental” Kit225STAT5Luc Cells (K/O is knockout). FIG. 2D: Shows a FACS profile of the Third CRISPR-Cas 9 Experiment Utilizing Pool 2-1 with Synthego K/O kit v2 prior to cell sorting to eliminate the few remaining CD25 positive cells. FIG. 2E: Compares the Kit225Stat5Luc CD25 K/O pool (final) FACS profile to the parental FACS profile. Note: FACS performed one month post cell sort; Clone 1-G9 isolated from this pool. FIG. 2F: Compares the Kit225Stat5Luc CD25 K/O Clone 1-G9 FACS profile to the parental FACS profile. FIG. 2G and FIG. 2H: both cell-based assays were run in parallel to characterize various IL-2 entities which also served to highlight the attributes of each assay. Kit225 STAT5Luc cells were used in FIG. 2G and Kit225 CD25K/O STAT5Luc cells were used in FIG. 2H. Aldesleukin, IL-2 Mutant A (Pegylated βγ-biased IL-2), Mutant B (βγ-biased IL-2), and IL-2 Mutant C (Pegylated aldesleukin) were used. FITC is fluorescin.

FIGS. 3A-3B. Selected optimization of IL-2 reporter assays. FIG. 3A: Dose response curve of IL-2 Mutant A at different treatment times. 5, 6, and 7-hour treatment time were shown in the plot. The table below is the summary of calculated parameters using four parameter logistic (4-PL) dose-response curve fit. Assay window (D/A) is also listed. FIG. 3B: Dose response curve of IL-2 Mutant A at difference cell plating time. 18, 19, and 20-hour cell plating time points were shown in the plot.

FIGS. 4A-4D. The pre-qualification study of the IL-2 reporter assay. FIG. 4A: A representative graph of the qualification study. It is a plot of 4-PL dose response curve of IL-2 Mutant A reference along with 200, 50, 35 and 100% four-target relative potency levels. FIG. 4B: All the relative potency data points, grouped by day, analyst, and target potency, were plotted. FIG. 4C: Residual plot of relative bias at each level of target potency. FIG. 4D: Shown is the linearity plot at all target potency levels using natural log scale to compare target potency level with calculated relative potency. Proportional bias (P_{bias}) table is shown below the plot.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, the term “interleukin-2” or “IL-2” as used herein, refers to any wild-type or native IL-2 from any vertebrate source, including mammals such as primates (e.g. humans) and rodents (e.g., mice and rats), unless otherwise indicated. The term encompasses unprocessed IL-2 as well as any mature form of IL-2 that lacks the N-terminal leader signal sequence. The term also encompasses naturally occurring variants of IL-2, e.g. splice variants or allelic variants. The amino acid sequence of mature human IL-2 is shown in SEQ ID NO: 6. Unprocessed human IL-2 additionally comprises an N-terminal 20 amino acid signal peptide, which is absent in the mature human IL-2 molecule. Human mature IL-2 has three cysteine residues, namely, C58, C105, and C125, of which C58 and C105 are linked intramolecularly by a disulfide bond (Tsuji et al., 1987, J. Biochem. 26: 129-134). Aldesleukin is a recombinant mature human IL-2 with a deletion of the N-terminal alanine residue (desAlal or desAl) and a substitution of serine for the cysteine at position 125 (C125S substitution) and expressed in E. coli has been found to be biologically active after in vitro refolding (Wang et al., 1984, Science, 224: 1431-1433; Yun et al., 1988, Kor. J. Biochem. 22: 120-126). This molecule has the nonproprietary name of aldesleukin, which comprises the amino acid sequence set forth in SEQ ID NO: 7.

As used herein, the term “control sequences” or “regulatory sequences” refers to nucleotide sequences necessary for the expression of an operably linked coding sequence in a particular host organism. The control sequences that are suitable for expression in eukaryotes, for example, include a transcription promoter, operator or enhancer sequences, response element, transcription termination sequences, and polyadenylation sequences for expression of a messenger RNA encoding a protein and a ribosome binding site for facilitating translation of the messenger RNA. Examples of control sequence include response and/or enhancer elements. STAT5 as used herein is an example of a control sequence that is a response element.

As used herein, the term “STAT5 response element” refers to a nucleotide sequence that binds a STAT5 dimer and which is located upstream of a transcription promoter operably linked to a nucleotide sequence of interest. STAT5 is a member of the signal transducer and activator of transcription factors (STAT) family, mediating growth and cytokine signaling. STAT5 consists of two closely related family members, STAT5A and STAT5B, which exhibit 96% sequence homology and are functionally redundant. Upon activation, STAT5 is phosphorylated by receptor tyrosine kinases and, in turn, forms homodimers or hererodimers with other family members through its SH2 domains. The dimerized STAT5 translocates to the nucleus and binds to the STAT5 response element (TTCNNNGAA, wherein each Nis independently any nucleotide) thereby activating an adjacent promoter to promote expression of an open reading frame located downstream of the promoter. In the present invention, the STAT5 response element comprises five copies of the 9-mer. SEQ ID NO: 1 is an example of a STAT5 response element comprising five copies of the 9-mer TTCTGAGAA.

As used herein, a nucleic acid is “operably linked” when it is placed into a functional relationship with another nucleic acid sequence, e.g., a regulatory sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “serial dilution” refers to the stepwise dilution of a substance in solution. Usually the dilution factor at each step is constant, resulting in a geometric progression of the concentration in a logarithmic fashion. A ten-fold serial dilution could be 1 M, 0.1 M, 0.01 M, 0.001 M, etc. Serial dilutions are used to accurately create highly diluted solutions as well as solutions for experiments resulting in concentration curves with a logarithmic scale. A tenfold dilution for each step is called a logarithmic dilution or log-dilution, a 3.16-fold (100.5-fold) dilution is called a half-logarithmic dilution or half-log dilution, and a 1.78-fold (100.25-fold) dilution is called a quarter-logarithmic dilution or quarter-log dilution. Serial dilutions are widely used in experimental sciences, including biochemistry, pharmacology, microbiology, and physics.

As used herein, the term “detectable polypeptide” refers to a polypeptide that may be detected using any method known in the art that is specific for detecting the polypeptide. A detectable polypeptide may be detected using an antibody specific for the polypeptide or an enzymatic assay that detects an activity of the polypeptide. For example, the detectable polypeptide may be luciferase, which may be detected by incubating the luciferase in the presence of its substrate luciferin and detecting fluorescence produced as the luciferase oxidizes the luciferin.

As used herein, the term “time sufficient” refers to the amount of time necessary to achieve a particular result or be able to detect or measure a particular result.

As used herein, the term “production batch” refers to a batch of finished product produced under good manufacturing practices (GMP) and intended for commercial release. The present invention provides an assay for determining potency of such a batch as a quality control step performed prior to release of said batch for commercial use.

The present invention provides a functional cell-based assay that can give a quantitative assessment of IL-2 mediated cellular signaling and the relative activity of engineered IL-2 analogs in a cell environment.

The present invention further provides a method for determining the potency of an interleukin 2 (IL-2) analog, comprising: (a) providing (i) a cell line that expresses an IL-2Rαβγ complex and a signal transducer and activator of transcription 5 (STAT5) signaling transduction pathway reporter comprising a STAT5 response element and a promoter linked to an open reading frame encoding a detectable polypeptide, and (ii) serial dilutions of an IL-2 analog; (b) contacting each serial dilution of the IL-2 analog with an aliquot of the cell line to provide a plurality of cultures; (c) incubating the cultures for a time sufficient to enable expression of the detectable polypeptide over time; and (d) measuring expression of the detectable polypeptide to determine the potency of the IL-2 analog.

In a further embodiment, the method further comprises comparing the potency of the IL-2 analog to the potency of a control IL-2 analog.

In a further embodiment, the potency of the control IL-2 analog is determined by (e) providing the cell line of step (a) and serial dilutions of the control IL-2 analog; (f) contacting each serial dilution of the control IL-2 analog with an aliquot of the cell line to provide a plurality of cultures; (g) incubating the cultures for a time sufficient for expression of the detectable polypeptide over time; and (h) measuring expression of the detectable polypeptide to determine the potency of the control IL-2 analog.

In a further embodiment, the cell line comprises Kit225 cells.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

In a further embodiment, the the control IL-2 analog comprises aldesleukin.

The present invention further provides a manufacturing process for producing a production batch of an interleukin 2 (IL-2) analog comprising the steps of: (a) synthesizing the IL-2 analog; (b) purifying the IL-2 analog; (c) formulating the IL-2 analog into a production batch; (d) obtaining a sample of the IL-2 analog from the production batch; (e) making a serial dilution of the IL-2 analog; (f) contacting each serial dilution of the IL-2 analog with an aliquot of a cell line that expresses (i) the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures; (g) incubating the cultures for a time sufficient for expression of the detectable polypeptide over time; and (f) measuring expression of the detectable polypeptide to determine the potency of the IL-2 analog biased.

In a further embodiment, the cell line comprises Kit225 cells.

In a further embodiment, the detectable polypeptide is a luciferase polypeptide.

In a further embodiment, the cell line comprises Kit225 cells.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

In a further embodiment, the control IL-2 analog comprises aldesleukin.

The present invention further provides a manufacturing process for producing a production batch of an interleukin 2 (IL-2) analog comprising the steps of: (a) synthesizing the IL-2 analog; (b) purifying the IL-2 analog; (c) formulating the IL-2 analog into a production batch; (d) obtaining a sample of the IL-2 analog from the production batch; (e) making a serial dilution of the IL-2 analog; (f) contacting each serial dilution of the IL-2 analog with an aliquot of a cell line that expresses (i) the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures; (g) incubating the cultures for a time sufficient for expression of the detectable polypeptide over time; and (f) measuring expression of the detectable polypeptide to determine the potency of the IL-2 analog biased.

In a further embodiment, the cell line comprises Kit225 cells.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

The present invention further provides a functional cell-based assay that can give a quantitative assessment of IL-2 mediated cellular signaling and the relative activity of engineered IL-2 analogs biased for the IL-2Rβγ complex compared to non-IL-2Rβγ biased IL-2 analogs in a cell environment that lacks expression of IL-2Rαβγ complexes. A cell line that expresses CD25, CD122, and CD132 was engineered to lack expression of CD25 and to comprise a signal transducer and activator of transcription 5 (STAT5) responsive reporter system comprising up to five copies of a STAT5 response element operably linked upstream to a promoter linked upstream to a detectable polypeptide reporter. The engineered cell line expresses the IL-2Rβγ complex and not the IL-2Rαβγ complex. The present invention enables the comparison of the relative impact of PEGylation and IL-2Rα blocking on IL-2 potency.

Thus, the present invention provides a method for determining the potency of an IL-2 analog biased for the IL-2Rβγ complex over the IL-2Rαβγ complex, comprising (a) providing (i) a cell line that expresses an IL-2Rβγ complex without expression of an IL-2Rαβγ complex and a STAT5 signaling transduction pathway reporter, which comprises a STAT5 response element and promoter linked to an open reading frame encoding a detectable polypeptide, and (ii) serial dilutions of an IL-2 analog biased for the IL-2Rβγ complex; (b) contacting each serial dilution of the IL-2 analog biased for the IL-2Rβγ complex with an aliquot of the cell line to provide a plurality of cultures; (c) incubating the cultures for a time sufficient for expression of the detectable polypeptide over time; and (d) measuring expression of the detectable polypeptide to determine the potency of the IL-2 analog biased for the IL-2Rβγ complex.

In a further embodiment, the method further comprises comparing the potency of an IL-2 analog biased for the IL-2Rβγ complex to the potency of an IL-2 analog capable of binding to an IL-2Rαβγ complex.

In a further embodiment, the potency of the IL-2 analog capable of binding to an IL-2Rαβγ complex is determined by (e) providing the cell line of step (a) above and serial dilutions of the IL-2 analog capable of binding to an IL-2Rαβγ complex; (f) contacting each serial dilution of the IL-2 analog capable of binding to an IL-2Rαβγ complex with an aliquot of the cell line to provide a plurality of cultures; (g) incubating the cultures for a time sufficient for expression of the detectable polypeptide over time; and (h) measuring expression of the detectable polypeptide to determine the potency of the IL-2 analog capable of binding to an IL-2Rαβγ complex.

In a further embodiment, the cell line comprises Kit225 cells, which have been modified to lack expression of the CD25 gene.

In a further embodiment, the detectable polypeptide is a luciferase polypeptide. An exemplary luciferase is encoded by an open reading frame comprising the nucleotide sequence set forth in SEQ ID NO: 2. In particular embodiments, the luciferase polypeptide is fused at the carboxy terminus to a destabilizing polypeptide. An example of a destabilizing polypeptide is a PEST polypeptide comprising an amino acid sequence rich in proline, glutamic acid, serine, and threonine. In particular embodiments, the PEST polypeptide in encoded by an open reading frame comprising the nucleotide sequence set forth in SEQ ID NO: 4, which is in-frame with the nucleotide sequence encoding the luciferase.

In a further embodiment, the STAT5 response element comprises one or more copies of the 9-mer nucleotide sequence TTCTGAGAA or TTCNNNGAA wherein each Nis independently any nucleotide or five copies of the 9-mer nucleotide sequence TTCTGAGAA as set forth in SEQ ID NO: 1. In particular embodiments, the STAT5 response element is operably linked to a mini promoter element or a promoter that requires a response element for initiating transcription. An example of a mini promoter element has the nucleotide sequence set forth in SEQ ID NO: 3.

In a further embodiment, the IL-2 analog capable of binding to an IL-2Rαβγ complex comprises aldesleukin.

In a further embodiment, the IL-2 analog biased for the IL-2Rβγ complex comprises at least one amino acid substitution or deletion that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises at least one non-natural amino acid substitution that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises one or more substitutions or deletions at a position selected from the group consisting of E15, H16, L19, D20, K34, T36, R37, T40, F41, K42, F43, Y44, E60, E61, K63, P64, E67, L71, D84, N88, V91, M103, C104, Y106, Q126, T123, and I129, wherein the amino acid positions correspond to the positions set forth in the amino acid sequence of SEQ ID NO: 6.

In a further embodiment, the non-natural amino acid is conjugated to a hydrophilic or hydrophobic polymer. In particular embodiments, the hydrophilic polymer is polyethylene glycol and the hydrophobic polymer is a fatty acid.

In a further embodiment, the STAT5 signaling transduction pathway reporter is provided by an expression vector comprising a nucleic acid molecule comprising one or more STAT5 response elements and minimal promoter operably linked to an open reading frame encoding the detectable polypeptide as set forth above. In a particular embodiment, the expression vector or fragment thereof is integrated into the genome of the cells comprising the cell line. In a particular embodiment, the expression vector persists in an autonomous state in the cells comprising the cell line.

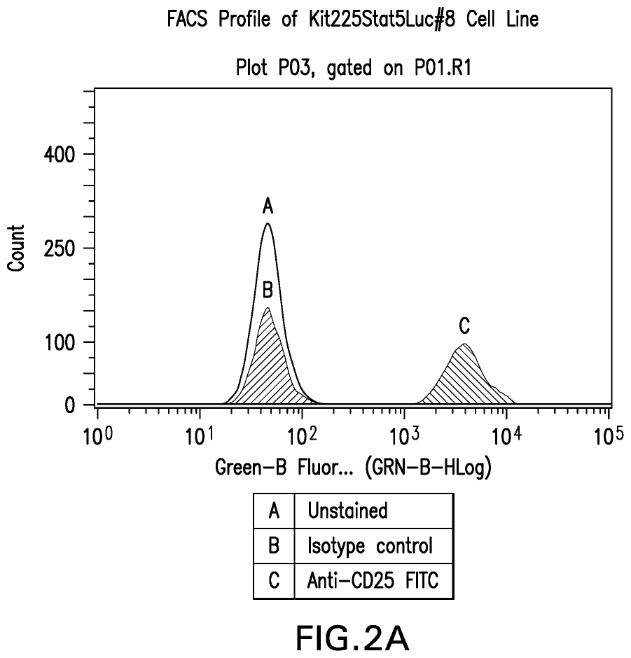

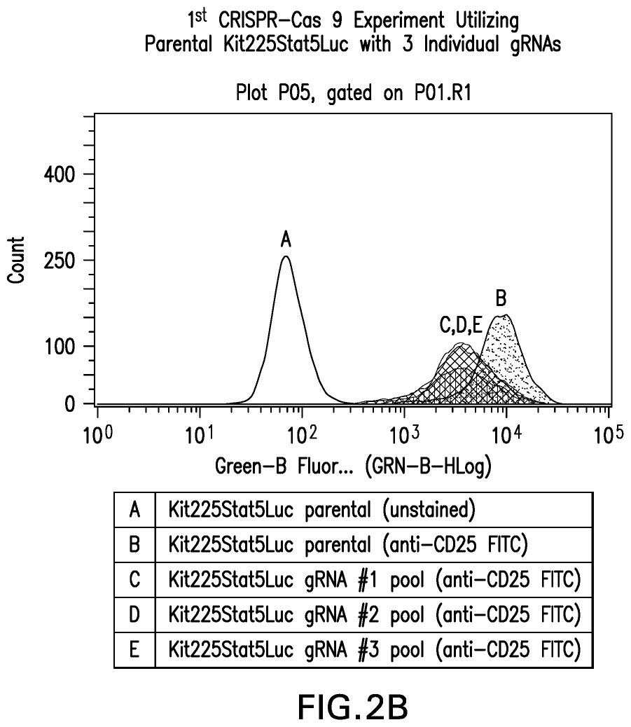

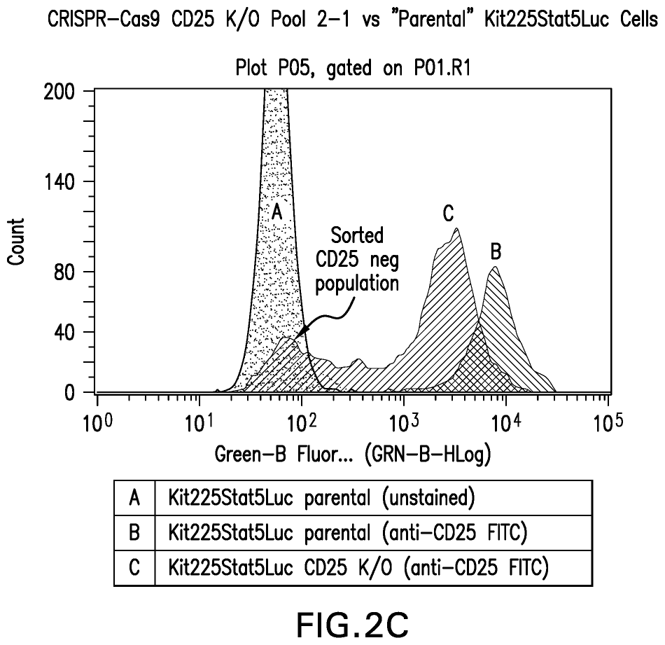

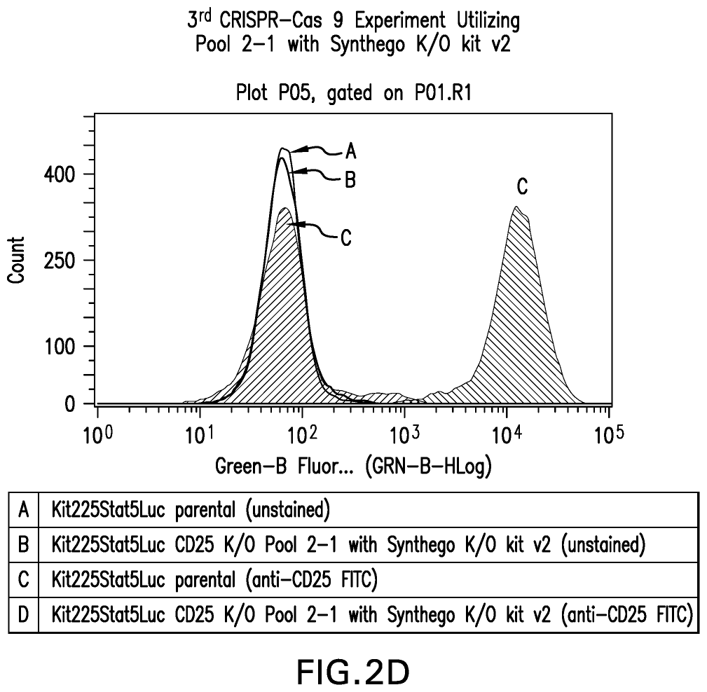

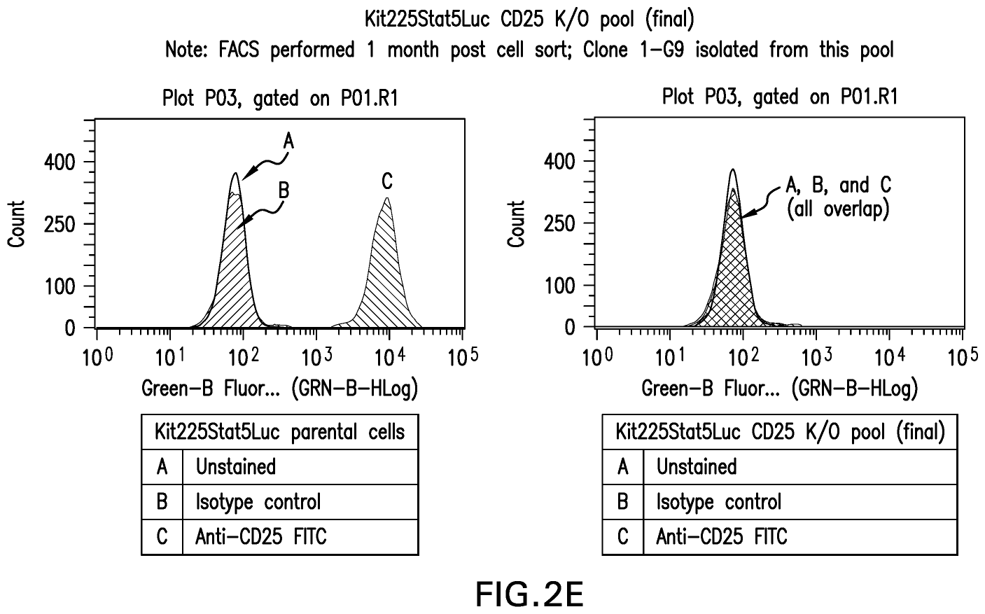

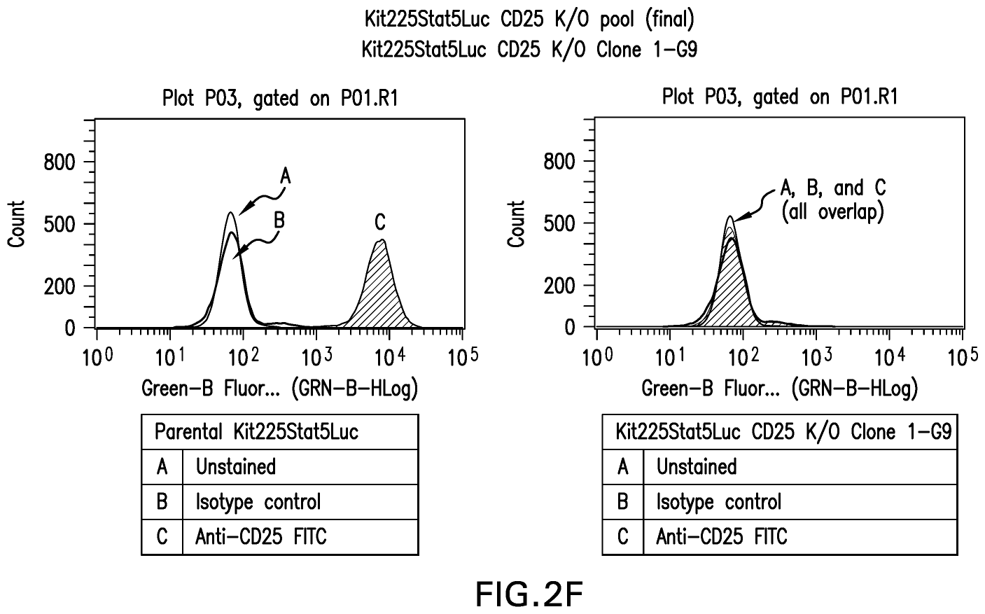

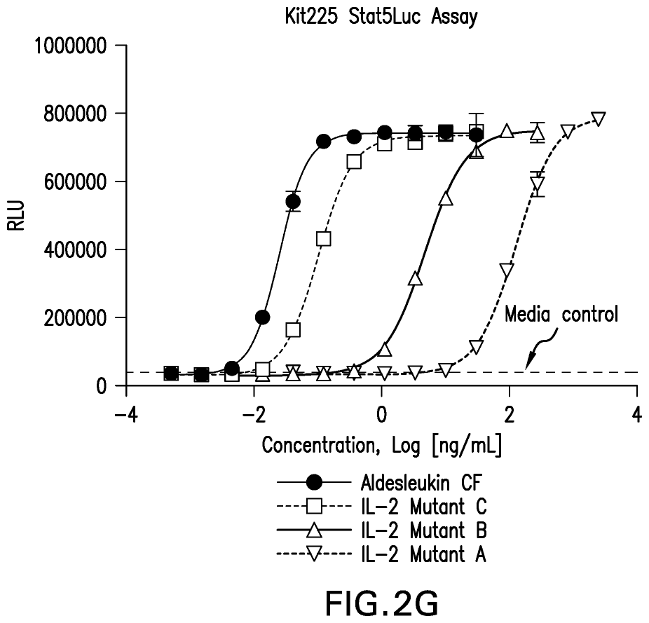

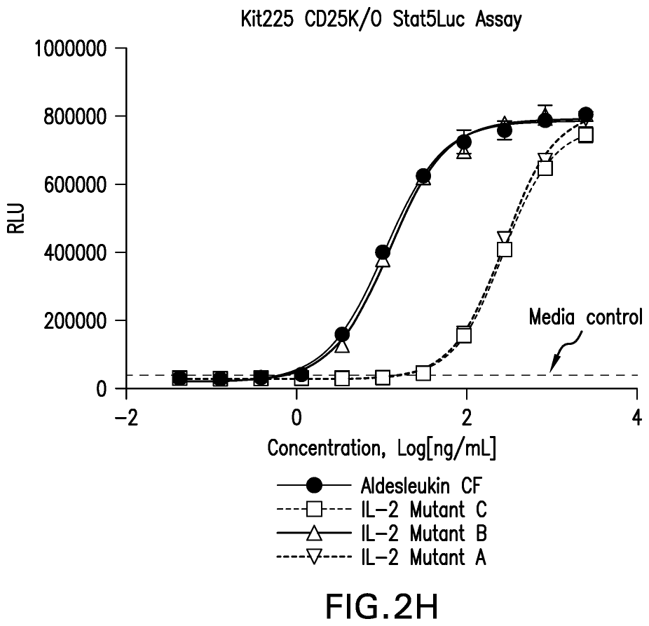

The present invention was exemplified using human T lymphocyte Kit225 cell line (Hori et al., Blood 70:1069-1072 (1987)) and engineering the cell line to comprise an exemplary vector comprising a STAT5 responsive luciferase reporter system. While Zumpe et al., Curr. Pharm. Biotechnol. 12: 1580-8 (2011), discloses a Kit225 cell line comprising a STAT5 reporter system designed for measuring potency of IL-7, the disclosed reporter system requires the destruction of the cells to measure potency. In contrast, the exemplary vector, pGL4.52[luc2P/STAT5 RE/Hygro] (See GenBank accession no. JX206457 (SEQ ID NO: 5); and U.S. Pat. Nos. 7,728,118 and 8,008,006), expresses a modified luciferase gene (luc2P) under the control of the STAT5 response element linked to a minimal promoter, which permits potency to be measured by detecting bioluminescence. The luc2P is encoded by an open reading frame encoding luc2 (SEQ ID NO: 2) fused in frame to an open reading frame encoding hPEST (SEQ ID NO: 4; a protein destabilization sequence disclosed in Yasanaga et al., J. Biotechnol. 194; 115-123 (2015)) to provide the luc2P. The hPEST allows luc2P protein levels to respond more quickly than those of luc2 to induction of transcription. The exemplary vector contains a STAT5 response element (STAT5 RE) (SEQ ID NO: 1) linked to a minimal promoter (SEQ ID NO: 3) that drives transcription of the detectable polypeptide reporter luc2P. Kit225 cells endogenously express the trimeric IL-2Rαβγ complex. To evaluate potency of IL-2 analogs with reduced or abrogated binding to the IL-2Rαβγ complex in an environment that provides the heterodimeric IL-2Rβγ receptor complex and not the IL-2Rαβγ complex, IL-2Rα (CD25) expression was eliminated from the Kit225 cell line using CRISPR/Cas9 technology, gene editing technology disclosed in U.S. Pat. Nos. 8,697,359 and 10,266,850, and commercially available in kits and services available world-wide. As exemplified in the examples, creation of a Kit225 cell line lacking CD25 expression and expressing a STAT5 responsive luciferase reporter has enabled the comparison of the relative impact of PEGylation and IL-2-Ra blocking muteins and demonstrated that in the absence of IL-2-Rα, the muteins had no impact on IL-2 potency (see FIG. 2D, for example).

As with all GMP products, biologics are required to undergo a rigorous regimen of release testing at the conclusion of manufacturing. Requirements will vary from product to product but generally will include certain specialized assays in addition to mandated compendial tests required of all injectable formulations. A key consideration for temperature-sensitive products is the coordination of sampling activities with the production process such that test samples are handled in a manner that is consistent with the bulk of the batch. This means they remain representative in all respects despite being separated physically from the main portion of the batch destined for patient administration. Thus, manufacturing requires making sure the potency of the biologic product remains consistent from lot to lot of the product.

Therefore, the present invention further provides a manufacturing process for producing a batch of an IL-2 analog biased for the IL-2Rβγ complex comprising the steps of synthesizing the IL-2 analog biased for the IL-2Rβγ complex, purifying the IL-2 biased for the IL-2Rβγ complex, and formulating the IL-2 biased for the IL-2Rβγ complex to provide a batch, wherein the improvement comprises (a) obtaining a sample of the IL-2 analog biased for the IL-2Rβγ from the process; (c) making a serial dilution of the IL-2 analog biased for the IL-2Rβγ complex; (d) contacting each serial dilution of the IL-2 analog biased for the IL-2Rβγ complex with an aliquot of a cell line that expresses (i) the IL-2Rβγ complex without expression of the IL-2Rαβγ complex and (ii) a STAT5 signaling transduction pathway reporter, which comprises a STAT5 response element and promoter linked to an open reading frame encoding a detectable polypeptide, to provide a plurality of cultures; (e) incubating the cultures for a time sufficient for expression of the detectable polypeptide over time; and (f) measuring expression of the detectable polypeptide to determine the potency of the IL-2 analog biased for the IL-2Rβγ complex. In particular embodiment batch is a production batch.

In a further embodiment, the cell line comprises Kit225 cells, which have been modified to lack expression of the CD25 gene.

In a further embodiment, the detectable polypeptide is a luciferase polypeptide. An exemplary luciferase is encoded by an open reading frame comprising the nucleotide sequence set forth in SEQ ID NO: 2. In particular embodiments, the luciferase polypeptide is fused at the carboxy terminus to a destabilizing polypeptide. An example of destabilizing polypeptide is a PEST polypeptide comprising an amino acid sequence rich in proline, glutamic acid, serine, and threonine. In particular embodiments, the PEST polypeptide in encoded by an open reading frame comprising the nucleotide sequence set forth in SEQ ID NO: 4, which is in-frame with the nucleotide sequence encoding the luciferase.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1. In particular embodiments, the STAT5 response is operably linked to a mini promoter element. An example of a mini promoter element has the nucleotide sequence set forth in SEQ ID NO: 3.

In a further embodiment, the IL-2 analog capable of binding to an IL-2Rαβγ complex comprises aldesleukin.

In a further embodiment, the IL-2 analog biased for the IL-2Rβγ complex comprises at least one amino acid substitution or deletion that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises at least one non-natural amino acid substitution that reduces or eliminates binding to the IL-2Rαβγ complex as determined by a Surface Plasmon Resonance (SPR) assay, which may be performed on a Biacore T200 (GE Healthcare) instrument.

In a further embodiment, the IL-2 analog biased for the IL-2Rβγ complex comprises one or more substitutions or deletions at a position selected from the group consisting of E15, H16, L19, D20, K34, T36, R37, T40, F41, K42, F43, Y44, E60, E61, K63, P64, E67, L71, D84, N88, V91, M103, C104, Y106, Q126, T123, and I129, wherein the amino acid positions correspond to the positions set forth in the amino acid sequence of SEQ ID NO: 6 (See for example, Suavé et al. PNAS USA 88: 4636 (1991); Charych et al. (NEKTAR), Clin Cancer Res 22(3): 680-690 (February 2016); WO9320849; WO2005086751; WO2008003473; WO2010085495; WO2019028419; WO2019028425; WO2020163532; WO2021030706; CN104231068A; U.S. Pat. Nos. 5,229,109; 9,206,243; 9,266,938; 9,428,567; 10183890; and 10610571).

In a further embodiment, the non-natural amino acid is conjugated to a hydrophilic or hydrophobic polymer. In particular embodiments, the hydrophilic polymer is polyethylene glycol and the hydrophobic polymer is a fatty acid.

In a further embodiment, the STAT5 signaling transduction pathway reporter is provided by an expression vector comprising a nucleic acid molecule comprising one or more STAT5 response elements and minimal promoter operably linked to an open reading frame encoding the detectable polypeptide as set forth above. In a particular embodiment, the expression vector or fragment thereof is integrated into the genome of the cells comprising the cell line. In a particular embodiment, the expression vector persists in an autonomous state in the cells comprising the cell line.

The present invention further provides a Kit225 cell comprising a nucleic acid molecule comprising a signal transducer and activator of transcription 5 (STAT5) response element and promoter operably linked to an open reading frame encoding a detectable polypeptide. In a further embodiment, the detectable polypeptide is a luciferase polypeptide. In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

The present invention further provides cell line comprising Kit225 cells comprising a nucleic acid molecule comprising a signal transducer and activator of transcription 5 (STAT5) response element and promoter operably linked to an open reading frame encoding a detectable polypeptide. In a further embodiment, the detectable polypeptide is a luciferase polypeptide. In a further embodiment, the STAT5 response element comprises one or more copies of the nucleotide sequence TCCNNNGAA wherein each Nis any nucleotide or five copies of the nucleotide sequence TTCTGAGAA as set forth in SEQ ID NO: 1.

The present further provides a method for producing a Kit225 cell that comprises a nucleic acid molecule comprising a STAT5 signaling transduction pathway reporter as disclosed herein comprising the steps of transfecting a Kit225 cell with an expression vector that comprises a STAT5 response element and promoter operably linked to an open reading frame encoding a detectable polypeptide and then deleting or disrupting the CD25 gene of said Kit225 cell to produce the Kit225 cell that comprises a nucleic acid molecule comprising a STAT5 signaling transduction pathway reporter.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

The present further provides a Kit225 cell modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

In a particular embodiment, the nucleic acid molecule is integrated into the genome of the Kit225 cell. In a particular embodiment, the nucleic acid molecule persists in an autonomous state in the Kit225 cell, e.g., in a plasmid capable of replicating and being maintained in a eukaryote cell.

The present invention further provides a cell line comprising Kit225 cells modified to lack expression of the CD25 gene and comprising a nucleic acid molecule comprising a STAT5 response element and promoter operably linked to an open reading frame encoding a detectable polypeptide.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

In a particular embodiment, the nucleic acid molecule is integrated into the genome of the Kit225 cells comprising the cell line. In a particular embodiment, the nucleic acid molecule persists in an autonomous state in the Kit225 cells comprising the cell line, e.g., in a plasmid capable of replicating and being maintained in a eukaryote cell.

In a further embodiment, the STAT5 signaling transduction pathway reporter is provided by an expression vector comprising a nucleic acid molecule comprising a STAT5 response element and promoter linked to an open reading frame encoding a detectable polypeptide. In a particular embodiment, the expression vector or fragment thereof is integrated into the genome of the cells comprising the cell line. In a particular embodiment, the expression vector persists in an autonomous state in the cells comprising the cell line.

The present further provides a method for producing a modified Kit225 cell that lacks expression of the CD25 gene and comprises a nucleic acid molecule comprising a STAT5 signaling transduction pathway reporter comprising (a) the steps of deleting or disrupting the CD25 gene of a Kit225 cell to produce a Kit225 cell that lacks CD25 expression and transfecting said Kit225 cell that lacks CD25 expression with an expression vector comprising a STAT5 response element and promoter operably linked to an open reading frame encoding a detectable polypeptide to produce the modified Kit225 cell; or, (b) the steps of transfecting a Kit225 cell with an expression vector that comprises a STAT5 response element and promoter operably linked to an open reading frame encoding a detectable polypeptide and then deleting or disrupting the CD25 gene of said Kit225 cell to produce the modified Kit225 cell.

In a further embodiment, the detectable polypeptide is a luciferase polypeptide.

In a further embodiment, the STAT5 response element comprises one or more copies of the nucleotide sequence TCCNNNGAA wherein each N is any nucleotide or five copies of the nucleotide sequence TTCTGAGAA as set forth in SEQ ID NO: 1.

The following examples are intended to promote a further understanding of the present invention.

### Example 1

### Material and Methods

### 1.1. Cell Line and Reagents

The human T lymphocyte cell line Kit225 was established by the lab of H. Uchino as described in the journal Blood 1987 volume 70: 1069-1072. The cells were maintained in culture media containing 10 ng/mL IL-2 (R&D Systems Cat #202-IL/CF), 10% Fetal Bovine Serum (HyClone Cat #SH30088.03), 1% HEPES buffer (Gibco Cat #15630-080), and 1% L-glutamine (Sigma Cat #G7513) in RPMI1640 basal media (Sigma Cat #R8758).

### 1.2. Generation of the Kit225 STAT5-Luc Stable Cell Line

The Kit225 STAT5-Luc cells were engineered in the following manner. The Kit225 parental cell line was transfected with the plasmid pGL4.52[luc2P/STAT5 RE/Hygro (Promega Part No. E465A lot #0000299955, GenBank Accession Number JX206457) using the 4D-Nucleofector Core Unit (Lonza Cat #AAF-1002B) and the SE Cell Line 4D-Nucleofector reagent kit (Lonza Cat #V4XC-1024). The STAT5 RE (STAT5 response element) comprises the nucleotide sequence set forth in SEQ ID NO: 1 and is operably linked to a mini promoter (nucleotide sequence of SEQ ID NO:3), which drives expression of an open reading frame (ORF) encoding a luciferase polypeptide (nucleotide sequence of SEQ ID NO:2) fused to a PEST degradation polypeptide from mouse ornithine (nucleotide sequence of SEQ ID NO:4). The transfection protocol provided by Lonza for Jurkat clone E6.1 cells was used in conjunction with an optimized pulse code for Kit225 cells. Briefly, for each reaction 1×10⁶ cells were centrifuged at 90×g for 10 minutes at room temperature. The cells were then resuspended in 100 μL freshly prepared complete nucleofector solution containing 2 ug plasmid DNA. The mixture was transferred to a nucleocuvette, placed within the 4D-Nucleofector Core Unit, and pulsed using the manufacturer's code CL-116. Afterwards the cells were gently resuspended, transferred to a 12-well tissue culture plate (Falcon Cat #353043) containing pre-warmed culture media, and placed in a tissue culture incubator set at 37° ° C., 5% CO₂. The resulting cell pools were placed under 0.6 mg/mL hygromycin B (Invitrogen Cat #10687010) selection after 72 hours and further expanded. The presence of the pSTAT5-Luc reporter was confirmed in a standard luciferase assay where cells were first treated with a titration of IL-2 followed by the addition of BrightGlo substrate (Promega Cat #E2620). Levels of luminescence, an indirect readout for luciferase activity, were measured using the Envision Multilabel plate reader (Perkin Elmer Model 2104-0010). Based upon reporter activity a cell pool was selected for single-cell cloning by limiting dilution which led to the isolation of Kit225 STAT5-Luc clone #8.

### 1.3. Generation of the (D25 K/O Kit225 STAT5-Luc Stable Cell Line

The CD25 K/O Kit225 STAT5-Luc cell line was established in the following manner. The Kit225 STAT5Luc clone #8 cells (described above section 1.2) were utilized in a CRISPR-Cas9 knockout (K/O) experiment testing three separate single-guide RNAs (sgRNAs) (#1=IL2RA+6026000, #2=IL2RA+6026003, #3=IL2RA+6026004) designed by Synthego Corporation at 3565 Haven Avenue, Suite 1, Menlo Park, CA 94025 USA, to target the human CD25 gene (also known as IL-2 alpha receptor gene).

The standard protocol provided by Synthego Corporation was followed. Briefly, sgRNAs were rehydrated in the provided nuclease free TE buffer (10 mM Tris-HCl, 1 mM EDTA, pH 8.0) to obtain a stock concentration of 100 μM. The sgRNAs were then further diluted in nuclease-free water to achieve a working concentration of 30 μM. For each sgRNA tested, a 6 μL volume of sgRNA was combined with 1 μL of Cas9 nuclease (Integrated DNA Technologies Cat #1081061), incubated 10 minutes at room temperature and then added to 1.5×10⁵ cells suspended in 23 μL of SE electroporation buffer (Lonza SE Cell Line 4D-Nucleofector reagent kit Cat #V4XC-1024). The mixture was transferred to a nucleocuvette strip, placed within the 4D-Nucleofector Core Unit (Lonza Cat #AAF-1002B), and pulsed using code CL-116 optimized for the Kit225 cells. Partial knockdown (but not complete knockout) of CD25 receptor expression was achieved with each separate sgRNA as determined by FACS analysis (Millipore Guava EasyCyte HT, anti-CD25 FITC-labeled Biolegend Cat #302616). The term FITC refers to fluorescin. In a second round of CRISPR-Cas9, each cell pool from the first experiment was treated separately with the two individual sgRNAs previously not utilized, thus generating pools annotated 1-2, 1-3, 2-1, 2-3, 3-1, and 3-2. These cell pools did contain small subpopulations of CD25 negative cells indicating the CD25 gene had been successfully knocked out, although the majority of cells still expressed high levels of CD25 receptor. However, when a third round of CRISPR-Cas9 was conducted utilizing pool 2-1 from the second experiment in conjunction with Synthego Corporation's K/O kit V2 (obtained through the Science Exchange platform, headquartered at 444 High Street, Suite 100, Palo Alto, CA 94301 USA), a cocktail comprised of three different sgRNAs directed against the human CD25 gene, approximately 95% knockout of CD25 receptor expression was achieved. The resulting cell pool was sorted on the BD FACSAria Fusion (Becton Dickinson) for CD25 negative cells which were placed in culture media containing IL-15 (R&D Systems Cat #247-ILB/CF) not IL-2. After expansion the pool of CD25 negative cells were single-cell cloned by limiting dilution to isolate CD25 K/O Kit225 STAT5-Luc clone 1-G9.

### 1.4 IL-2 Cell-Based Reporter Assay

The functional cell-based assay was optimized using CD25K/O Kit225STAT5Luc clone 1G9, engineered with a phosphoSTAT5 promoter linked to the luciferase reporter gene and CD25 (IL-2Rα) knockout. IL-2 mutant A is a pegylated aldesleukin analog that further comprises mutations in the region important for binding to the IL-2Rα that abrogate binding to the IL-2Rα and the IL-2Rαβγ complex. The mutant binds to IL-2Rβγ complex with intermediate affinity and regulates T-cell activation and downstream phosphorylation of STAT5 with luminescence produced after incubation with a luciferase substrate. IL-2 mutant B is a non-pegylated version of IL-2 Mutant A. IL-2 mutant C is the pegylated aldesleukin analog that lacks the mutations of Mutant A and thus binds the IL-2Rα, the IL-2Rβγ complex, and the IL-2Rαβγ complex. The PEGylated IL-2 analogs were all conjugated to the same polyethylene glycol (PEG) polymer at the same position within the polypeptide sequence.

CD25K/O Kit225STAT5Luc clone 1G9 (CD25K/O Kit225) are cultured in RPMI 1640 Medium (RPMI 1640 Medium, GlutaMAX™, HEPES) containing 10% Heat Inactivated Fetal Bovine Serum, 100 U/mL Penicillin Streptomycin, 600 μg/mL Hygromycin B and 20 ng/ml recombinant human IL-15. CD25K/O Kit225 are sub-cultured in freshly supplemented rhIL-15 medium, after centrifugation and removal of old medium, at a concentration between 0.5×10⁵ and 1.2×10⁵ cells/mL. Cells are incubated in a 37° C.+5% CO₂ humidified incubator and grown for 2-3 days undisturbed. The approximate doubling time is 24 hours. For the assay, 45-50 mL of CD25K/O Kit225 are harvested and centrifuged at 300×g at room temperature for two minutes to pellet cells. Supernatant is removed and cells are resuspended in RPMI 1640 Medium (RPMI 1640 Medium, GlutaMAX™, HEPES) containing 2% Heat Inactivated Fetal Bovine Serum, 100 U/mL Penicillin Streptomycin, 600 μg/mL Hygromycin B. CD25K/O cells are counted, and plates are seeded at a volume of 50 μL/well with a concentration of 1.0×10⁴ cells/well in a 96-well tissue culture plate. Cell plates are placed in a humidified incubator set at 37° C. and 5% CO₂ overnight for 18+1 hours. On day two of the assay, IL-2 Mutant A serial dilution is prepared in a dilution block. Preparation of standards and controls are diluted in assay media containing RPMI 1640 Medium (RPMI 1640 Medium, GlutaMAX™, HEPES) containing 2% Heat Inactivated Fetal Bovine Serum, 100 U/mL Penicillin Streptomycin, 600 μg/mL Hygromycin B. For each assay plate, dilutions are prepared in singleton and tested in duplicate on each plate. Standards and controls are prepared at twice the final concentration, 80 μg/mL, and a four-fold serial dilution is performed over 8 dilutions. The assay plate accommodates for cell control wells containing assay media only. After serial dilution of IL-2 mutant A is complete, CD25K/O Kit225 cell plates are removed from overnight incubation. 50 μL of serially diluted IL-2 mutant A is transferred to the cell plate to the corresponding wells, cell plates are tapped gently for mixing. CD25K/O Kit225 assay plate is returned to a humidified incubator set at 37° C. and 5% CO₂ for 5 hours #15 minutes. After incubation, One-GLO™ luciferase substrate is equilibrated to room temperature and 100 μL One-GLO™ is added to the assay plate. Downstream phosphorylation of STAT5 produced after incubation with a luciferase substrate is then measured using PerkinElmer ENVISION Plate reader.

### 1.5. Statistical Analysis

The main objective of the pre-qualification study is to estimate the assay accuracy, intermediate precision, and linearity across the normal operating range of the assay conditions following the methods described in USP<1033> Biological Assay Validation, U.S. Pharmacopoeia 2010. All analyses were based on the natural logarithmic transformation on the relative potency values. Geometric mean, percent relative bias, percent geometric standard deviation (% GSD), and percent relative standard deviation (% RSD) were calculated using formulas from USP <1033>. All statistical analysis was carried out using JMP® version 13 software (SAS Institute, Cary, NC).

### Example 2

### 2.1. Generation of the Kit225 STAT5-Luc Stable Cell Line

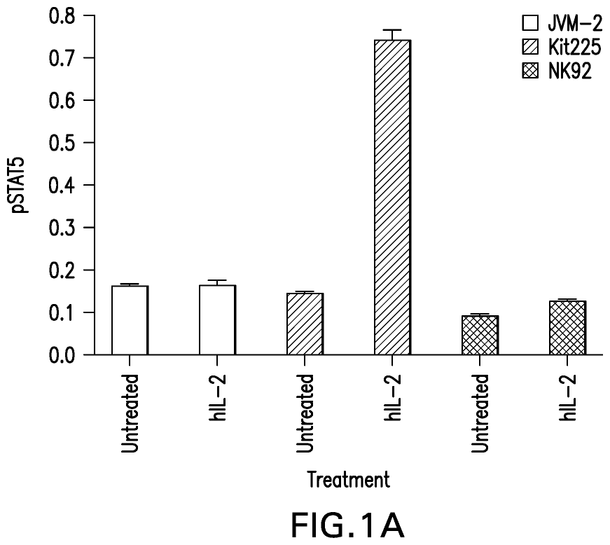

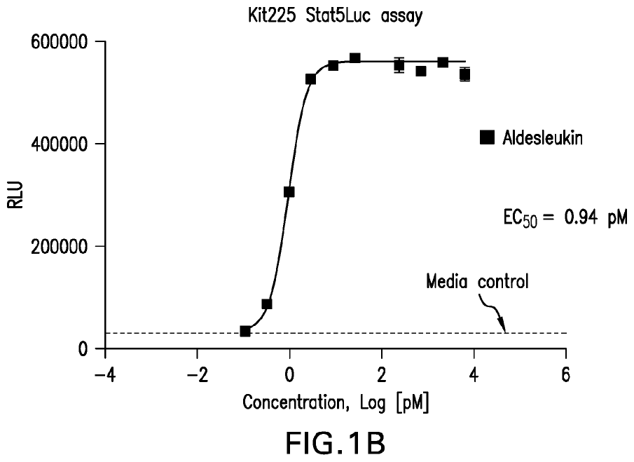

The Kit225 cell line originally obtained from the lab of H. Uchino (Kyoto University, Japan) was identified among several cell lines tested to generate a strong pSTAT5 signal when treated with IL-2 (FIG. 1A). For this reason, the Kit225 cell line was selected for engineering with a commercially available pSTAT5-Luc reporter plasmid. This was executed using the SE Cell Line 4D-Nucleofector reagent kit, an established transfection protocol for Jurkat clone E6.1 cells with an optimized pulse code for Kit225 cells, and the 4D-Nucleofector Core Unit, all obtained from Lonza. Of the four resulting cell pools which were cultured under hygromycin B selection, three had confirmed luciferase activity upon IL-2 treatment indicating the cells had been successfully transfected. Single-cell cloning by dilution was then performed on a chosen cell pool from which Kit225 STAT5-Luc clone #8 was isolated. Integral to developing an IL-2 responsive cell-based assay, the utility of the engineered Kit225 STAT5-Luc #8 cell line was initially demonstrated in a dose-response experiment with aldesleukin CF (FIG. 1B).

### 2.2. Generation of the CD25 K/O Kit225 STAT5-Luc Stable Cell Line

Subsequent to the establishment of the Kit225 STAT5-Luc #8 cell line (described in Section 2.1) it was desirable to knockout CD25 receptor expression of these cells (FIG. 2A). This was achieved using CRISPR-Cas9 technology in conjunction with several different sgRNAs used in a sequential manner. In an initial knockout experiment, the resulting cell pools were shown by FACS analysis to have reduced levels of CD25 receptor expression but notably not a complete knock out of the CD25 gene (FIG. 2B). However, in a second round of CRISPR-Cas9 in which cell pools from the first attempt were used with sgRNAs in a sequential manner, minor subpopulations of CD25 negative cells were generated as well as a further overall reduction of CD25 receptor expression (FIG. 2C). Finally, in a third round of CRISPR-Cas9 using a cocktail of sgRNAs and one pool from the second experiment, approximately 95% of the cells were negative for CD25 receptor expression (FIG. 2D). The resulting cell pool was processed using the BD FACSAria Fusion cell sorter to obtain a pure population of CD25 negative cells. Immediately these cells were placed in culture media containing IL-15 rather than IL-2 to mitigate the risk of any CD25 positive cell contaminant which could over time outcompete the CD25 negative cells. After expansion the CD25 negative cell population was reanalyzed by FACS to confirm the absence of CD25 positive cells (FIG. 2E). These cells were then single-cell cloned by limiting dilution to isolate the CD25 K/O Kit225 STAT5-Luc clone 1-G9 (FIG. 2F). In a similar manner to the “parental” Kit225 STAT5-Luc cell line, the CD25 K/O Kit225 Stat4-Luc cell line was used to establish a second IL-2 responsive cell-based assay.

Both cell-based assays were run in parallel to characterize various IL-2 entities which also served to highlight the attributes of each assay. The assay utilizing the “parental” Kit225 STAT5-Luc cells exquisitely distinguishes between aldesleukin CF (wt. IL-2) and a βγ-biased form of IL-2 (IL-2 Mutant B) as well as their pegylated forms (Mutants A and C, respectively), each entity producing a unique dose-response curve (FIG. 2G). The companion assay utilizing the CD25 K/O Kit225 STAT5-Luc cells elegantly demonstrates the similarity in potency of aldesleukin CF (wt. IL-2) and the βγ-biased IL-2 (IL-2 Mutant B) or for comparison their pegylated forms, in the absence of the CD25 receptor (IL-2 receptor alpha) (FIG. 2H). As expected, the βγ-bias effect has been lost with the curves for aldesleukin and IL-2 Mutant B now overlapping. Only the effect of PEGylation serves to right-shift the curves, to equal extent, demonstrated by the overlapping curves of Mutant C (pegylated aldesleukin variant) and IL-2 Mutant A (βγ-biased IL-2 pegylated).

### 2.3. IL-2 Cell-Based Reporter Assay Optimization

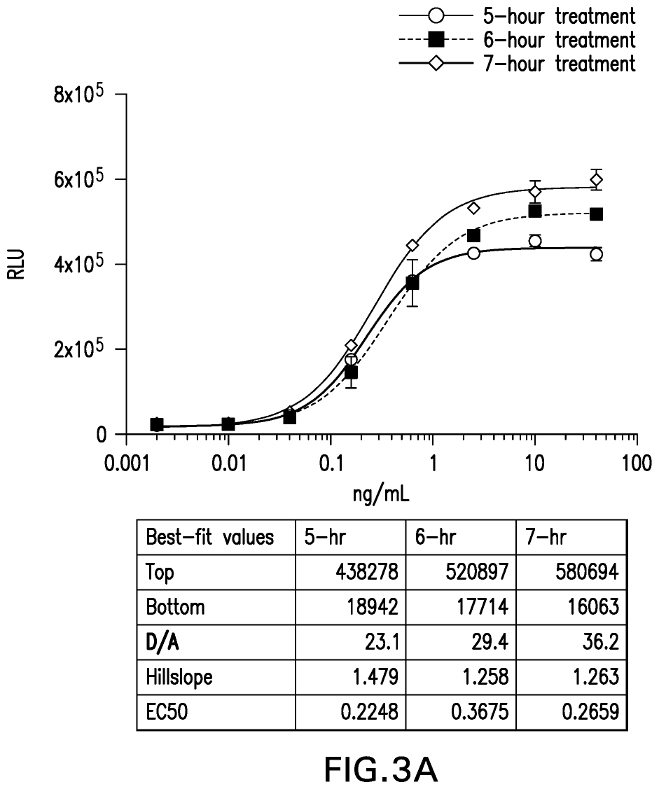

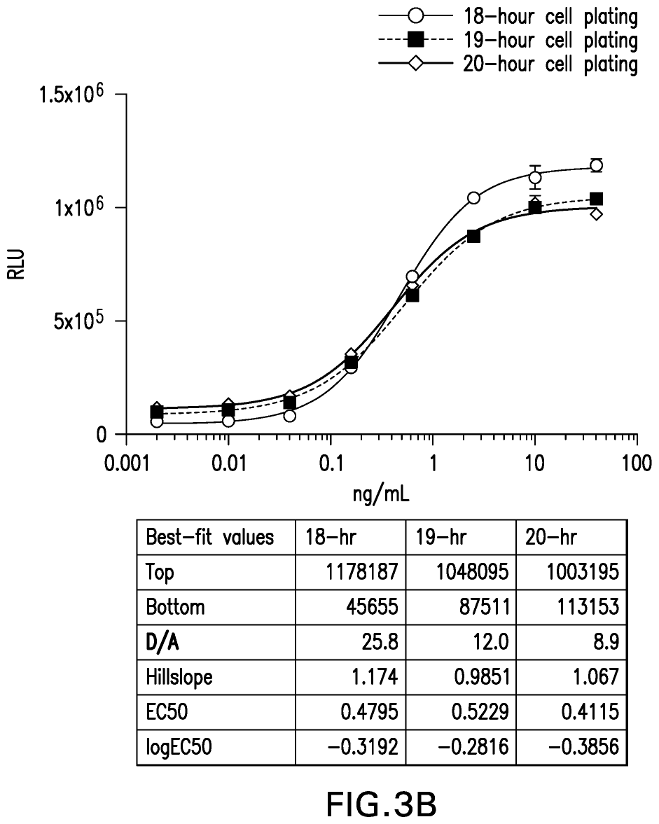

The IL-2 reporter assay using CD25K/O Kit225STAT5Luc clone 1G9 cells were further optimized for sample testing. The early chosen optimized condition is to plate cells overnight in a 96-well plate, cells were than treated with IL-2 entities for around six hours before adding luciferase substrate for detection. The treatment time was then optimized further. The five-, six-, and seven-hour treatment times were tested side-by-side. As shown in FIG. 3A, longer treatment time does seem to increase assay window (D/A) without significant shift in EC₅₀. The six-hour treatment time was chosen due to slight better assay accuracy (data not shown) and more practical handling time for an analyst. Even a five-hour treatment time is a viable choice due to significant high assay window observed in this assay. Cell plating time was also evaluated. As shown in FIG. 3B, 18, 19 and 20-hour cell plating time were shown here. An 18-hour cell plating time seems to give significantly higher assay window. An 18-hour cell plating time was then chosen. Further evaluation suggested that an 18+1 hour cell plating time is a viable option for analyst without sacrificing much assay performance (data not shown). The final optimized assay condition is summarized in Table 1.

TABLE 1
Summary of assay optimization

| Parameters | Current Setting | Note |
|---|---|---|
| Cell density | 10,000 cells/well | Different cell densities tested |
| | | but no significant difference |
| | | in performance observed. |
| Cell Passage # | At least passage 30 | Not tested > p30 |
| Drug treatment | 6 hours | 5-7 hours treatment time are |
| time | | all acceptable |
| Plate type | Solid white wall 96-well | Solid white plates gave 3-fold |
| | tissue culture-treated | increase in signal with lower |
| | plates | variability |
| Assay medium | RPMI-1640 with HEPES | RMPI-1640 supplemented |
| | (25 mM), GlutaMAX ™ | with HEPES (10 mM) and |
| | | L-Glutamine |
| Cell incubation | 18 hours overnight | 18 ± 1 hours make no |
| before | incubation | difference |
| treatment | | |
| Plate reader | EnVision from Perkin | SpectraMax M5/M5e gave |
| | Elmer | low signal, not preferred. |

### 2.4. IL-2 Cell-Based Assay Pre-Qualification

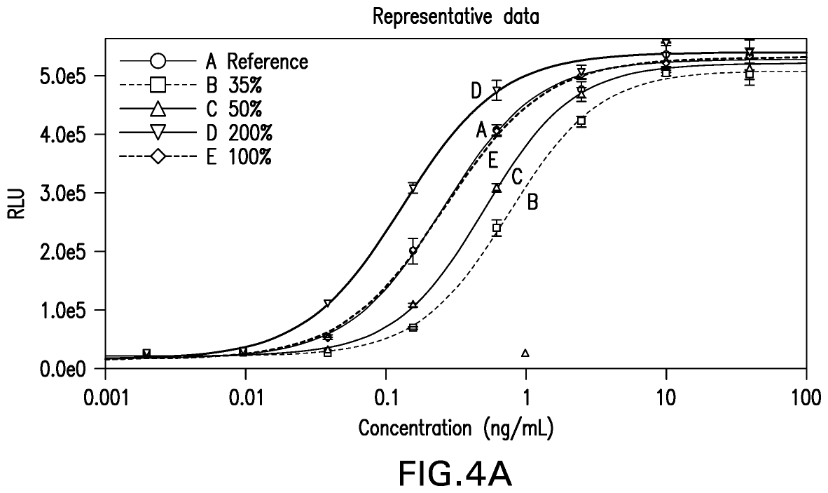

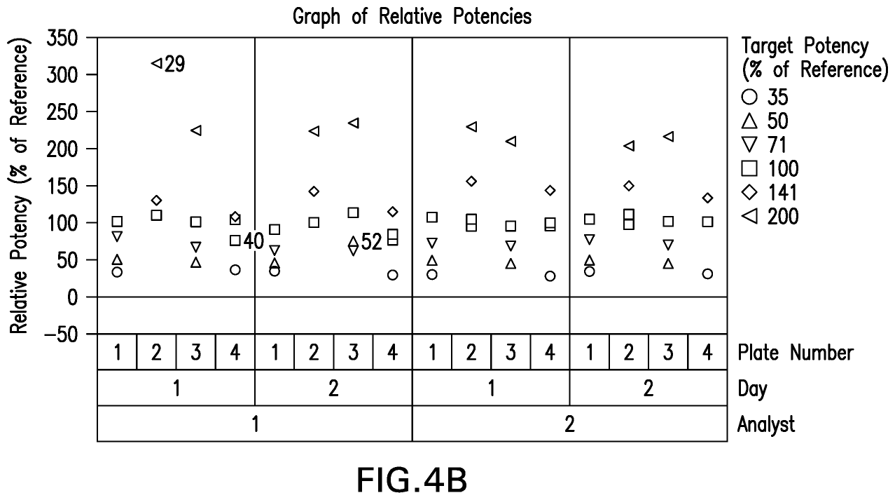

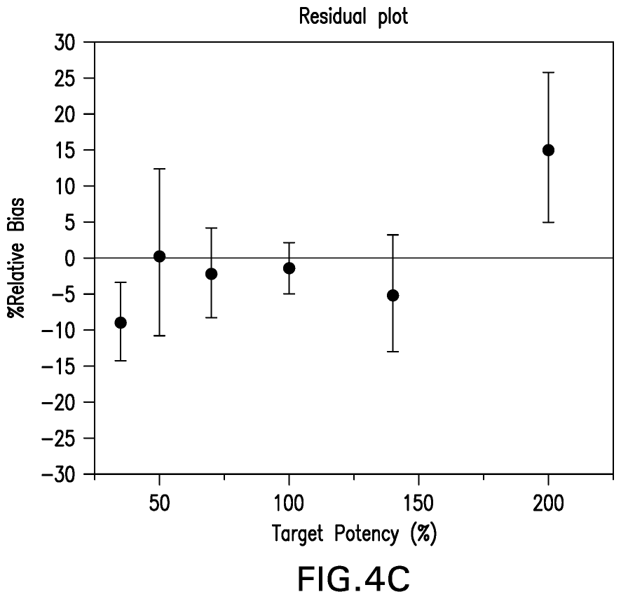

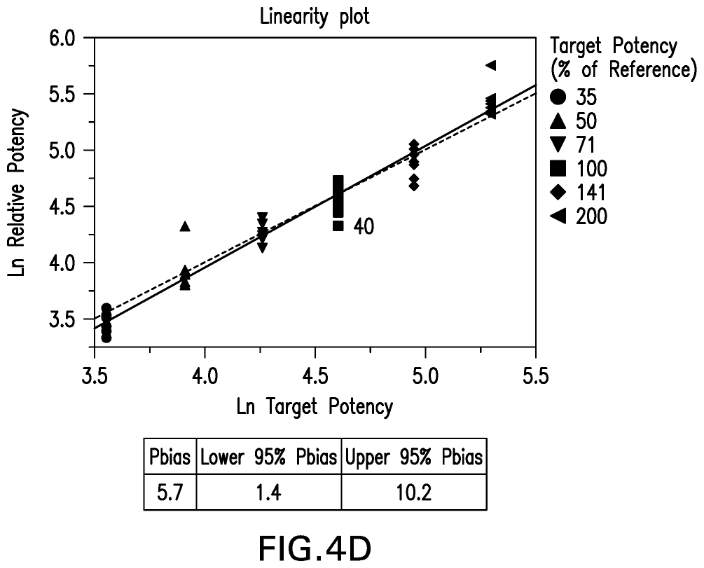

A pre-qualification study of this cell-based assay was performed to assess the following performance characteristics of the method: relative accuracy, precision, linearity and range. A pre-qualification study is similar to a qualification study except that it is performed in a non-GMP laboratory. Five potency doses were tested at a range of 35% to 200% of IL-2 Mutant A reference material (35%, 50%, 71%, 100%, 141%, and 200% relative potency levels) in a total of 16 plates. Each potency dose was tested by two analysts, with four independent runs (days) by one analyst and two independent runs (days) by the other analyst. 1-4 independent replicates of the same dilution were performed in each run. Since the 100% target potency is the most important value and can be used to evaluate intraplate precision, higher repeat (N=24 compared to N=12 for other potency levels) was performed. Also, IL-2 Mutant A drug substance/drug product samples will be tested at this concentration, making it most relevant. A representative graph of the dose-response curve results in the pre-qualification study is shown in FIG. 4A. All the relative potency data points, grouped by day, analyst and target potency, were plotted in FIG. 4B.

### 2.4.1. Relative Accuracy

Relative Accuracy, expressed as Relative bias, between the target relative potency of the dilution sample and the measured relative potency (geometric mean (GM) of relative potency (RP) of replicate samples) was calculated at individual levels of the dilutional linearity experiment using the formula:

$$\% Relative bias = 100 * ( relative potency measured / target relative potency - 1 ) \qquad ( Eq . 1 )$$

The relative bias plot is shown in FIG. 4C and summarized in Table 2.

TABLE 2
Summary of assay accuracy and bias
for different target potency levels

| | | | % | Lower 90% | Upper 90% |
|---|---|---|---|---|---|
| Target Relative | | Geometric | Relative | Relative | Relative |
| Potency (%) | N | mean | Bias | Bias | Bias |
| 35 | 8 | 32 | −9 | −14.3 | −3.4 |
| 50 | 8 | 50 | 0.2 | −10.7 | 12.4 |
| 71 | 8 | 69 | −2.3 | −8.3 | 4.1 |
| 100 | 24 | 99 | −1.5 | −5 | 2.2 |
| 141 | 8 | 134 | −5.2 | −13 | 3.3 |
| 200 | 8 | 230 | 14.9 | 5 | 25.8 |

Within the testing range of 35% and 200%, all measured geometric means of the six potency levels have good correlation with their target relative potency with % relative bias ranging from −9.0% to 14.9%.

### 2.4.2 Linearity and Range of Reliable Response

Linearity refers to the assays' ability to generate proportional results. This can be achieved through the calculation of proportional bias, which is related to the slope (β) from the regression of log (relative potency) on log (target potency), see Coffey et al., BioProcess International, 11: 42-49 (2013). The formula is given in Equation 2.

$$\% Proportional Bias = 100 \times ( {2}^{β - 1} - 1 ) \qquad ( Eq . 2 )$$

For assessing linearity, target potency values (based on dilution of IL-2 Mutant A reference material) were plotted against measured relative potency values (relative potency values for individual replicates or Geometric mean of relative potency) on a natural log scale. Regression analysis was performed and the overall coefficient of determination R2, intercept, slope, proportional trend bias (%), 95% confidence interval on P_{Bias} and the regression line are reported in FIG. 4D. As shown in FIG. 4D, the regression line generated within the tested range of 35% to 200% relative potency has a slope of 1.08 with a proportional trend bias of 5.7%. In this case, implying that the estimated 2-fold increase in observed potency is more than expected for a perfectly linear assay and within range of experience for a bioassay. The bias was reported per 2-fold dilution since the 2-fold is a common scale. The data suggests that there is a good linear relationship within the tested range. Combined with relative accuracy data on each dilution level, we conclude that the data within the tested range of 35% to 200% is reliable in this assay.

### 2.4.3. Intermediate Precision

Intermediate precision (IP) is the overall variability from analysts, days, and plates. In evaluating precision, the relative potencies were first log-transformed to satisfy the requirements of normality and variance homogeneity. Variance component analysis was carried out on the log-transformed relative potencies by performing a mixed-model analysis with restricted maximum likelihood estimation (REML) using JMP 13.0. Precision estimates were determined for each target potency level separately as well as overall. In the latter analysis, to account for the systematic effect due to testing samples with different potencies, the potency level was treated as a fixed linear covariate in the model. The variance estimates (s2) are converted back to the original units and expressed in terms of % RSD (relative standard deviation) and % GSD (geometric standard deviation) using the following formulas given in Equation 3.

$$\% RSD = 100 \times \sqrt{{e}^{{s}^{2}} - 1} ; \% GSD = 100 \times ( {e}^{s} - 1 ) \qquad ( Eq . 3 )$$

The estimated % RSD and % GSD of the variance component analyses are summarized in Table 3.

TABLE 3
Variability estimates at each target potency level

| Target Relative | | Lower | Upper | |
|---|---|---|---|---|
| Potency (%) | % RSD | 95% RSD | 95% RSD | % GSD |
| 35 | 9 | 5.9 | 18.4 | 9.4 |
| 50 | 17.3 | 11.4 | 36.1 | 18.8 |
| 71 | 9.5 | 6.2 | 19.4 | 9.9 |
| 100 | 10.4 | 8.1 | 14.6 | 10.9 |
| 141 | 12.9 | 8.5 | 26.5 | 13.7 |
| 200 | 13.6 | 8.9 | 28 | 14.5 |

The overall percent geometric standard deviation (% GSD, intermediate precision) for a target concentration of 100% was 10.9% and the % GSD across different concentration levels was less than 20%.

TABLE of Sequences

| SEQ | | |
|---|---|---|
| ID | | |
| NO: | Description | Sequence |
| 1 | STAT5 Response | agttctgagaaaagtagttctgagaaaagtagttctgagaaaagtagttctgagaaaagt |
| | Element | agttctgagaaaagt |
| | comprising five | |
| | copies of the 9- | |
| | mer having the | |
| | nucleotide | |
| | sequence | |
| | TTCTGAGAA | |
| 2 | DNA encoding | atggaagatgccaaaaacattaagaagggcccagcgccattctacccactcgaagac |
| | Luc2 codon- | gggaccgccggcgagcagctgcacaaagccatgaagcgctacgccctggtgcccg |
| | optimized for | gcaccatcgcctttaccgacgcacatatcgaggtggacattacctacgccgagtacttc |
| | expression in | gagatgagcgttcggctggcagaagctatgaagcgctatgggctgaatacaaaccatc |
| | human cells | ggatcgtggtgtgcagcgagaatagcttgcagttcttcatgcccgtgttgggtgccctgt |
| | | tcatcggtgtggctgtggccccagctaacgacatctacaacgagcgcgagctgctgaa |
| | | cagcatgggcatcagccagcccaccgtcgtattcgtgagcaagaaagggctgcaaaa |
| | | gatcctcaacgtgcaaaagaagctaccgatcatacaaaagatcatcatcatggatagca |
| | | agaccgactaccagggcttccaaagcatgtacaccttcgtgacttcccatttgccaccc |
| | | ggcttcaacgagtacgacttcgtgcccgagagcttcgaccgggacaaaaccatcgcc |
| | | ctgatcatgaacagtagtggcagtaccggattgcccaagggcgtagccctaccgcac |
| | | cgcaccgcttgtgtccgattcagtcatgcccgcgaccccatcttcggcaaccagatcat |
| | | ccccgacaccgctatcctcagcgtggtgccatttcaccacggcttcggcatgttcacca |
| | | cgctgggctacttgatctgcggctttcgggtcgtgctcatgtaccgcttcgaggaggag |
| | | ctattcttgcgcagcttgcaagactataagattcaatctgccctgctggtgcccacactat |
| | | ttagcttcttcgctaagagcactctcatcgacaagtacgacctaagcaacttgcacgaga |
| | | tcgccagcggggggcgccgctcagcaaggaggtaggtgaggccgtggccaaacg |
| | | cttccacctaccaggcatccgccagggctacggcctgacagaaacaaccagcgccat |
| | | tctgatcacccccgaaggggacgacaagcctggcgcagtaggcaaggtggtgccctt |
| | | cttcgaggctaaggtggtggacttggacaccggtaagacactgggtgtgaaccagcg |
| | | cggcgagctgtgcgtccgtggccccatgatcatgagcggctacgttaacaaccccga |
| | | ggctacaaacgctctcatcgacaaggacggctggctgcacagcggcgacatcgccta |
| | | ctgggacgaggacgagcacttcttcatcgtggaccggctgaagagcctgatcaaatac |
| | | aagggctaccaggtagccccagccgaactggagagcatcctgctgcaacaccccaa |
| | | catcttcgacgccggggtcgccggcctgcccgacgacgatgccggcgagctgcccg |
| | | ccgcagtcgtcgtgctggaacacggtaaaaccatgaccgagaaggagatcgtggact |
| | | atgtggccagccaggttacaaccgccaagaagctgcgcggtggtgttgtgttcgtgga |
| | | cgaggtgcctaaaggactgaccggcaagttggacgcccgcaagatccgcgagattct |
| | | cattaaggccaagaagggcggcaagatcgccgtg |
| 3 | Mini promoter | gagggtatataatggaagctcgacttccag |
| | element | |
| 4 | DNA encoding | tctcacggcttccctcccgaggtggaggagcaggccgccggcaccctgcccatgag |
| | PEST degradation | ctgcgcccaggagagcggcatggatagacaccctgctgcttgcgccagcgccagga |
| | sequence from | tcaacgtc |
| | mouse ornithine; | |
| | codon-optimized | |
| | for expression in | |
| | human cells | |
| 5 | JX206457.1 | actcgtcctttttcaatattattgaagcatttatcagggttactagtacgtctctcaaggata |
| | Reporter vector | agtaagtaatattaaggtacgggaggtattggacaggccgcaataaaatatctttattttc |
| | pGL4.52[luc2P/ | attacatctgtgtgttggttttttgtgtgaatcgatagtactaacatacgctctccatcaaaa |
| | STAT5 RE/Hygro], | caaaacgaaacaaaacaaactagcaaaataggctgtccccagtgcaagtgcaggtgc |
| | complete sequence | cagaacatttctctggcctaactggccggtacctgagctcagttctgagaaaagtagttc |
| | 285-359-encodes | tgagaaaagtagttctgagaaaagtagttctgagaaaagtagttctgagaaaagtctcg |
| | STAT5 | aggatatcaagatctggcctcggcggccaagcttagacactagagggtatataatgga |
| | 405-435-nuc seq | agctcgacttccagcttggcaatccggtactgttggtaaagccaccatggaagatgcca |
| | of miniP | aaaacattaagaagggcccagcgccattctacccactcgaagacgggaccgccggc |
| | 468-2243 | gagcagctgcacaaagccatgaagcgctacgccctggtgcccggcaccatcgccttt |
| | encodes Luc2P | accgacgcacatatcgaggtggacattacctacgccgagtacttcgagatgagcgttc |
| | (includes PEST) | ggctggcagaagctatgaagcgctatgggctgaatacaaaccatcggatcgtggtgtg |
| | | cagcgagaatagcttgcagttcttcatgcccgtgttgggtgccctgttcatcggtgtggc |
| | | tgtggccccagctaacgacatctacaacgagcgcgagctgctgaacagcatgggcat |
| | | cagccagcccaccgtcgtattcgtgagcaagaaagggctgcaaaagatcctcaacgt |
| | | gcaaaagaagctaccgatcatacaaaagatcatcatcatggatagcaagaccgactac |
| | | cagggcttccaaagcatgtacaccttcgtgacttcccatttgccacccggcttcaacga |
| | | gtacgacttcgtgcccgagagcttcgaccgggacaaaaccatcgccctgatcatgaac |
| | | agtagtggcagtaccggattgcccaagggcgtagccctaccgcaccgcaccgcttgt |
| | | gtccgattcagtcatgcccgcgaccccatcttcggcaaccagatcatccccgacaccg |
| | | ctatcctcagcgtggtgccatttcaccacggcttcggcatgttcaccacgctgggctact |
| | | tgatctgcggctttcgggtcgtgctcatgtaccgcttcgaggaggagctattcttgcgca |
| | | gcttgcaagactataagattcaatctgccctgctggtgcccacactatttagcttcttcgct |
| | | aagagcactctcatcgacaagtacgacctaagcaacttgcacgagatcgccagcggc |
| | | ggggcgccgctcagcaaggaggtaggtgaggccgtggccaaacgcttccacctacc |
| | | aggcatccgccagggctacggcctgacagaaacaaccagcgccattctgatcacccc |
| | | cgaaggggacgacaagcctggcgcagtaggcaaggtggtgcccttcttcgaggcta |
| | | aggtggtggacttggacaccggtaagacactgggtgtgaaccagcgcggcgagctgt |
| | | gcgtccgtggccccatgatcatgagcggctacgttaacaaccccgaggctacaaacg |
| | | ctctcatcgacaaggacggctggctgcacagcggcgacatcgcctactgggacgag |
| | | gacgagcacttcttcatcgtggaccggctgaagagcctgatcaaatacaagggctacc |
| | | aggtagccccagccgaactggagagcatcctgctgcaacaccccaacatcttcgacg |
| | | ccggggtcgccggcctgcccgacgacgatgccggcgagctgcccgccgcagtcgtcg |
| | | tgctggaacacggtaaaaccatgaccgagaaggagatcgtggactatgtggccagccaggtt |
| | | acaaccgccaagaagctgcgcggtggtgttgtgttcgtggacgaggtgcctaaaggactgac |
| | | cggcaagttggacgcccgcaagatccgcgagattctcattaaggccaagaagggcggcaag |
| | | atcgccgtgaattctcacggcttccctcccgaggtggaggagcaggccgccggcaccctgccc |
| | | atgagctgcgcccaggagagcggcatggatagacaccctgctgcttgcgccagcgccaggat |
| | | caacgtctaaggccgcgactctagagtcggggcggccggccgcttcgagcagacatg |
| | | ataagatacattgatgagtttggacaaaccacaactagaatgcagtgaaaaaaatgcttt |
| | | atttgtgaaatttgtgatgctattgctttatttgtaaccattataagctgcaataaacaagtta |
| | | acaacaacaattgcattcattttatgtttcaggttcagggggaggtgtgggaggttttttaa |
| | | agcaagtaaaacctctacaaatgtggtaaaatcgataaggatccgtttgcgtattgggc |
| | | gctcttccgctgatctgcgcagcaccatggcctgaaataacctctgaaagaggaacttg |
| | | gttagctaccttctgaggcggaaagaaccagctgtggaatgtgtgtcagttagggtgtg |
| | | gaaagtccccaggctccccagcaggcagaagtatgcaaagcatgcatctcaattagtc |
| | | agcaaccaggtgtggaaagtccccaggctccccagcaggcagaagtatgcaaagca |
| | | tgcatctcaattagtcagcaaccatagtcccgcccctaactccgcccatcccgccccta |
| | | actccgcccagttccgcccattctccgccccatggctgactaattttttttatttatgcaga |
| | | ggccgaggccgcctctgcctctgagctattccagaagtagtgaggaggcttttttggag |
| | | gcctaggcttttgcaaaaagctcgattcttctgacactagcgccaccatgaagaagccc |
| | | gaactcaccgctaccagcgttgaaaaatttctcatcgagaagttcgacagtgtgagcga |
| | | cctgatgcagttgtcggagggcgaagagagccgagccttcagcttcgatgtcggcgg |
| | | acgcggctatgtactgcgggtgaatagctgcgctgatggcttctacaaagaccgctac |
| | | gtgtaccgccacttcgccagcgctgcactacccatccccgaagtgttggacatcggcg |
| | | agttcagcgagagcctgacatactgcatcagtagacgcgcccaaggcgttactctcca |
| | | agacctccccgaaacagagctgcctgctgtgttacagcctgtcgccgaagctatggat |
| | | gctattgccgccgccgacctcagtcaaaccagcggcttcggcccattcgggccccaa |
| | | ggcatcggccagtacacaacctggcgggatttcatttgcgccattgctgatccccatgt |
| | | ctaccactggcagaccgtgatggacgacaccgtgtccgccagcgtagctcaagccct |
| | | ggacgaactgatgctgtgggccgaagactgtcccgaggtgcgccacctcgtccatgc |
| | | cgacttcggcagcaacaacgtcctgaccgacaacggccgcatcaccgccgtaatcga |
| | | ctggtccgaagctatgttcggggacagtcagtacgaggtggccaacatcttcttctggc |
| | | ggccctggctggcttgcatggagcagcagactcgctacttcgagcgccggcatcccg |
| | | agctggccggcagccctcgtctgcgagcctacatgctgcgcatcggcctggatcagct |
| | | ctaccagagcctcgtggacggcaacttcgacgatgctgcctgggctcaaggccgctg |
| | | cgatgccatcgtccgcagcggggccggcaccgtcggtcgcacacaaatcgctcgcc |
| | | ggagcgcagccgtatggaccgacggctgcgtcgaggtgctggccgacagcggcaa |
| | | ccgccggcccagtacacgaccgcgcgctaaggaggtaggtcgagtttaaactctaga |
| | | accggtcatggccgcaataaaatatctttattttcattacatctgtgtgttggttttttgtgtgt |
| | | tcgaactagatgctgtcgaccgatgcccttgagagccttcaacccagtcagctccttcc |
| | | ggtgggcgcggggcatgactatcgtcgccgcacttatgactgtcttctttatcatgcaact |
| | | cgtaggacaggtgccggcagcgctcttccgcttcctcgctcactgactcgctgcgctcggtcgt |
| | | tcggctgcggcgagcggtatcagctcactcaaaggcggtaatacggttatccacagaatcag |
| | | gggataacgcaggaaagaacatgtgagcaaaaggccagcaaaaggccaggaaccgtaaa |
| | | aaggccgcgttgctggcgtttttccataggctccgcccccctgacgagcatcacaaaaatcga |
| | | cgctcaagtcagaggtggcgaaacccgacaggactataaagataccaggcgtttccccctgg |
| | | aagctccctcgtgcgctctcctgttccgaccctgccgcttaccggatacctgtccgcctttctccc |
| | | ttcgggaagcgtggcgctttctcatagctcacgctgtaggtatctcagttcggtgtaggtcgttc |
| | | gctccaagctgggctgtgtgcacgaaccccccgttcagcccgaccgctgcgccttatccggta |
| | | actatcgtcttgagtccaacccggtaagacacgacttatcgccactggcagcagccactggta |
| | | acaggattagcagagcgaggtatgtaggcggtgctacagagttcttgaagtggtggcctaac |
| | | tacggctacactagaagaacagtatttggtatctgcgctctgctgaagccagttaccttcggaa |
| | | aaagagttggtagctcttgatccggcaaacaaaccaccgctggtagcggtggtttttttgtttg |
| | | caagcagcagattacgcgcagaaaaaaaggatctcaagaagatcctttgatcttttctacgg |
| | | ggtctgacgctcagtggaacgaaaactcacgttaagggattttggtcatgagattatcaaaaa |
| | | ggatcttcacctagatccttttaaattaaaaatgaagttttaaatcaatctaaagtatatatga |
| | | gtaaacttggtctgacagcggccgcaaatgctaaaccactgcagtggttaccagtgcttgatc |
| | | agtgaggcaccgatctcagcgatctgcctatttcgttcgtccatagtggcctgactccccgtcgt |
| | | gtagatcactacgattcgtgagggcttaccatcaggccccagcgcagcaatgatgccgcgag |
| | | agccgcgttcaccggcccccgatttgtcagcaatgaaccagccagcagggagggccgagcg |
| | | aagaagtggtcctgctactttgtccgcctccatccagtctatgagctgctgtcgtgatgctaga |
| | | gtaagaagttcgccagtgagtagtttccgaagagttgtggccattgctactggcatcgtggtat |
| | | cacgctcgtcgttcggtatggcttcgttcaactctggttcccagcggtcaagccgggtcacatg |
| | | atcacccatattatgaagaaatgcagtcagctccttagggcctccgatcgttgtcagaagtaa |
| | | gttggccgcggtgttgtcgctcatggtaatggcagcactacacaattctcttaccgtcatgcca |
| | | tccgtaagatgcttttccgtgaccggcgagtactcaaccaagtcgttttgtgagtagtgtatac |
| | | ggcgaccaagctgctcttgcccggcgtctatacgggacaacaccgcgccacatagcagtactt |
| | | tgaaagtgctcatcatcgggaatcgttcttcggggcggaaagactcaaggatcttgccgctat |
| | | tgagatccagttcgatatagcccactcttgcacccagttgatcttcagcatcttttactttcacc |
| | | agcgtttcggggtgtgcaaaaacaggcaagcaaaatgccgcaaagaagggaatgagtgcg |
| | | acacgaaaatgttggatgctcat |
| 6 | IL-2 (mature) | APTSSSTKKTQLQLEHLLLDLQMILNGINNYKNPKLTR |
| | | MLTFKFYMPKKATELKHLQCLEEELKPLEEVLNLAQS |
| | | KNFHLRPRDLISNINVIVLELKGSETTFMCEYADETATI |
| | | VEFLNRWITFCQSIISTLT |
| 7 | Aldesleukin | PTSSSTKKTQLQLEHLLLDLQMILNGINNYKNPKLTRM |
| | | LTFKFYMPKKATELKHLQCLEEELKPLEEVLNLAQSKN |
| | | FHLRPRDLISNINVIVLELKGSETTFMCEYADETATIVEF |
| | | LNRWITFSQSIISTLT |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for determining the potency of an interleukin 2 (IL-2) analog biased for the IL-2 receptor beta-gamma (IL-2Rβγ) complex over the IL-2 receptor alpha-beta-gamma (IL-2Rαβγ) complex, comprising:
(a) providing (i) a cell line that expresses an IL-2Rβγ complex without expression of an IL-2Rαβγ complex and a signal transducer and activator of transcription 5 (STAT5) signaling transduction pathway reporter comprising a STAT5 response element and a promoter linked to an open reading frame encoding a detectable polypeptide, and (ii) serial dilutions of an IL-2 analog biased for the IL-2Rβγ complex;
(b) contacting each serial dilution of the IL-2 analog biased for the IL-2Rβγ complex with an aliquot of the cell line to provide a plurality of cultures;
(c) incubating the cultures for a time sufficient to enable expression of the detectable polypeptide over time; and
(d) measuring expression of the detectable polypeptide to determine the potency of the IL-2 analog biased for the IL-2Rβγ complex.

2. The method of claim 1, wherein the method further comprises comparing the potency of the IL-2 analog biased for the IL-2Rβγ complex to the potency of a control IL-2 analog, which comprises an IL-2 analog capable of binding to an IL-2Rαβγ complex.

3. The method of claim 2, wherein the potency of the IL-2 analog capable of binding to an IL-2Rαβγ complex is determined by
(e) providing the cell line of step (a) and serial dilutions of the IL-2 analog capable of binding to an IL-2Rαβγ complex;
(f) contacting each serial dilution of the IL-2 analog capable of binding to an IL-2Rαβγ complex with an aliquot of the cell line to provide a plurality of cultures;
(g) incubating the cultures for a time sufficient for expression of the detectable polypeptide over time; and
(h) measuring expression of the detectable polypeptide to determine the potency of the IL-2 analog capable of binding to an IL-2Rαβγ complex.

4. The method of claim 1, wherein the cell line comprises Kit225 cells, which have been modified to lack expression of the CD25 gene.

5-10. (canceled)

11. A method for producing an interleukin 2 (IL-2) analog biased for the IL-2 receptor beta-gamma (IL-2Rβγ) complex over the IL-2 receptor alpha-beta-gamma (IL-2Rαβγ) complex, comprising:
(a) providing an IL-2 analog capable of binding the IL-2Rαβγ complex;
(b) substituting one or more amino acids of the IL-2 analog that are at the interface between the IL-2 and the IL-2Rα of the IL-2Rαβγ complex with a natural amino acid or non-natural amino acid to provide an IL-2 analog biased for the IL-2Rβγ complex;
(c) making a serial dilution of the IL-2 analog biased for the IL-2Rβγ complex;
(d) contacting each serial dilution of the IL-2 analog biased for the IL-2Rβγ complex with an aliquot of a cell line that expresses (i) the IL-2Rβγ complex without expression of the IL-2Rαβγ complex and (ii) a signal transducer and activator of transcription 5 (STAT5) signaling transduction pathway reporter comprising a STAT5 response element and promoter linked to an open reading frame encoding a detectable polypeptide, to provide a plurality of cultures;
(e) incubating the cultures for a time sufficient to enable expression of the detectable polypeptide over time;
(f) measuring expression of the detectable polypeptide to determine the potency of the IL-2 analog biased for the IL-2Rβγ complex; and
(g) isolating the IL-2 analog biased for the IL-2Rβγ complex if the potency of the IL-2 analog biased for the IL-2Rβγ complex has a potency within a predetermined range.

12. The method of claim 11, wherein the potency is substantially the same as the potency of an IL-2 analog capable of binding the IL-2Rαβγ complex.

13. The method of claim 12, wherein the potency of the IL-2 analog capable of binding the IL-2Rαβγ complex is determined by
(h) making a serial dilution of the IL-2 analog capable of binding the IL-2Rαβγ complex;
(i) contacting each serial dilution of the IL-2 analog capable of binding the IL-2Rαβγ complex with an aliquot of a cell line that expresses (x) the IL-2Rβγ complex without also expressing the IL-2Rαβγ complex and (y) a signal transducer and activator of transcription 5 (STAT5) signaling transduction pathway reporter comprising a STAT5 response element and promoter linked to an open reading frame encoding a detectable polypeptide, to provide a plurality of cultures;
(j) incubating the cultures for a time sufficient for expression of the detectable polypeptide over time; and
(k) measuring expression of the detectable polypeptide to determine the potency of the IL-2 analog capable of binding the IL-2Rαβγ complex.

14-18. (canceled)

19. A manufacturing process for producing a production batch of an interleukin 2 (IL-2) analog biased for the IL-2 receptor beta-gamma (IL-2Rβγ) complex comprising the steps of:
(a) synthesizing the IL-2 analog biased for the IL-2Rβγ complex;
(b) purifying the IL-2 analog biased for the IL-2Rβγ complex;
(c) formulating the IL-2 analog biased for the IL-2Rβγ complex into a production batch;
(d) obtaining a sample of the IL-2 analog biased for the IL-2Rβγ from the production batch;
(e) making a serial dilution of the IL-2 analog biased for the IL-2Rβγ complex;
(f) contacting each serial dilution of the IL-2 analog biased for the IL-2Rβγ complex with an aliquot of a cell line that expresses (i) the IL-2Rβγ complex without expression of the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures;
(g) incubating the cultures for a time sufficient for expression of the detectable polypeptide over time; and
(h) measuring expression of the detectable polypeptide to determine the potency of the IL-2 analog biased for the IL-2Rβγ complex.

20. The method of claim 19, wherein the cell line comprises Kit225 cells, which have been modified to lack expression of the CD25 gene.

21-22. (canceled)

23. A Kit225 cell modified to lack expression of the CD25 gene and comprising a nucleic acid molecule comprising a signal transducer and activator of transcription 5 (STAT5) response element and promoter operably linked to an open reading frame encoding a detectable polypeptide.

24. The Kit225 cell of claim 23, wherein the detectable polypeptide is a luciferase polypeptide.

25. The Kit225 cell of claim 23, wherein the STAT5 response element comprises one or more copies of the nucleotide sequence TCCNNNGAA wherein each N is any nucleotide or the nucleotide sequence as set forth in SEQ ID NO: 1.

26. A cell line comprising Kit225 cells modified to lack expression of the CD25 gene and comprising a nucleic acid molecule comprising a signal transducer and activator of transcription 5 (STAT5) response element and promoter operably linked to an open reading frame encoding a detectable polypeptide.

27-28. (canceled)

29. A method for determining the potency of an interleukin 2 (IL-2) analog, comprising:
(a) providing (i) a cell line that expresses an IL-2Rαβγ complex and a signal transducer and activator of transcription 5 (STAT5) signaling transduction pathway reporter comprising a STAT5 response element and a promoter linked to an open reading frame encoding a detectable polypeptide, and (ii) serial dilutions of an IL-2 analog;
(b) contacting each serial dilution of the IL-2 analog with an aliquot of the cell line to provide a plurality of cultures;
(c) incubating the cultures for a time sufficient to enable expression of the detectable polypeptide over time; and
(d) measuring expression of the detectable polypeptide to determine the potency of the IL-2 analog.

30. The method of claim 29, wherein the method further comprises comparing the potency of the IL-2 analog to the potency of a control IL-2 analog.

31. The method of claim 29, wherein the potency of the control IL-2 analog is determined by
(e) providing the cell line of step (a) and serial dilutions of the control IL-2 analog;
(f) contacting each serial dilution of the control IL-2 analog with an aliquot of the cell line to provide a plurality of cultures;
(g) incubating the cultures for a time sufficient for expression of the detectable polypeptide over time; and
(h) measuring expression of the detectable polypeptide to determine the potency of the control IL-2 analog.

32. The method of claim 29, wherein the cell line comprises Kit225 cells.

33-35. (canceled)

36. A manufacturing process for producing a production batch of an interleukin 2 (IL-2) analog comprising the steps of:
(a) synthesizing the IL-2 analog;
(b) purifying the IL-2 analog;
(c) formulating the IL-2 analog into a production batch;
(d) obtaining a sample of the IL-2 analog from the production batch;
(e) making a serial dilution of the IL-2 analog;
(f) contacting each serial dilution of the IL-2 analog with an aliquot of a cell line that expresses (i) the IL-2Rαβγ complex and (ii) a signal transducer and activator of transcription 5 (STAT5) signaling transduction pathway reporter, which comprises a STAT5 response element and promoter linked to an open reading frame encoding a detectable polypeptide, to provide a plurality of cultures;
(g) incubating the cultures for a time sufficient for expression of the detectable polypeptide over time; and
(h) measuring expression of the detectable polypeptide to determine the potency of the IL-2 analog biased.

37. The method of claim 36, wherein the cell line comprises Kit225 cells.

38-42. (canceled)

43. A cell line comprising Kit225 cells comprising a nucleic acid molecule comprising a signal transducer and activator of transcription 5 (STAT5) response element and promoter operably linked to an open reading frame encoding a detectable polypeptide.

44-45. (canceled)
